(19) United States
(12) Patent Application Publication
(10) Pub. No.: US 2004/0128720 A1
(43) Pub. Date: Jul. 1, 2004

(54) Plants characterized by enhanced growth and methods and nucleic acid constructs useful for generating same

(76) Inventors: Aaron Kaplan, Jerusalem (IL); Judy Lieman-Hurwitz, Jerusalem (IL); Daniella Schatz, Jerusalem (IL); Ron Mittler, Jerusalem (IL); Shimon Rachmilevitch, Ramat Gan (IL)

Correspondence Address: c/o ANTHONY CASTORINA, G.E. EHRLICH (1995) LTD., SUITE 207, 2001 JEFFERSON DAVIS HIGHWAY, ARLINGTON, VA, 22202, US

(21) Appl. No.: 10/669,174

(22) Filed: Sep. 24, 2003

Related U.S. Application Data
(63) Continuation-in-part of application No. 10/410,432, filed on Apr. 10, 2003.
Application No. 10/410,432 is a continuation-in-part of application No. PCT/IL02/00250, filed on Mar. 26, 2002.
(63) Continuation-in-part of application No. 09/887,038, filed on Jun. 25, 2001, now abandoned.
Application No. 09/887,038 is a continuation of application No. 09/332,041, filed on Jun. 14, 1999, now Pat. No. 6,320,101.

(51) Int. Cl. A01H 1/00; C12N 15/82

(52) U.S. Cl. 800/287; 800/294; 435/468

(57) ABSTRACT

A method of enhancing growth and/or commercial yield of a plant is provided. The method is effected by expressing within the plant a polypeptide including an amino acid sequence at least 60% homologous to that set forth in SEQ ID NOs: 3, 5, 6, 7, 10, 11, 12 or 13.

This application is a continuation-in-part of U.S. patent application Ser. No. 10/410,432, filed Apr. 10, 2003, which is a continuation-in-part of PCT/IL02/00250, filed Mar. 26, 2002, which claims priority of U.S. patent application Ser. No. 09/828,173, filed Apr. 9, 2001.

This application is also a continuation-in-part of U.S. patent application Ser. No. 09/887,038, filed Jun. 25, 2001, which is a continuation of U.S. patent application Ser. No. 09/332,041, filed Jun. 14, 1999, now U.S. Pat. No. 6,320,101, issued Nov. 20, 2001.

### FIELD AND BACKGROUND OF THE INVENTION

The present invention relates to plants characterized by enhanced growth and to methods and nucleic acid constructs useful for generating same.

Growth and productivity of crop plants are the main parameters of concern to a commercial grower. Such parameters are affected by numerous factors including the nature of the specific plant and allocation of resources within it, availability of resources in the growth environment and interactions with other organisms including pathogens.

Growth and productivity of most crop plants are limited by the availability of CO₂ to the carboxylating enzyme ribulose 1,5-bisphosphate carboxylase/oxygenase (Rubisco). Such availability is determined by the ambient concentration of CO₂ and stomatal conductance, and the rate of CO₂ fixation by Rubisco as determined by the Km(CO₂) and Vmax of this enzyme [31-33].

In C3 plants, the concentration of CO₂ at the site of Rubisco is lower than the Km(CO₂) of the enzyme, particularly under water stress conditions. As such, these crop plants exhibit a substantial decrease in growth and productivity when exposed to low CO₂ conditions induced by, for example, stomatal closure which can be caused by water stress.

Many photosynthetic microorganisms are capable of concentrating CO₂ at the site of Rubisco to thereby overcome the limitation imposed by the low affinity of Rubisco for CO₂ [34].

Higher plants of the C4 and the CAM physiological groups can also raise the concentration of CO₂ at the site of Rubisco by means of dual carboxylations which are spatially (in C4) or temporally (in CAM) separated.

Since plant growth and productivity especially in C3 crop plants are highly dependent on CO₂ availability to Rubisco and fixation rates, numerous attempts have been made to genetically modify plants in order to enhance CO₂ concentration or fixation therein in hopes that such modification would lead to an increase in growth or yield.

As such, numerous studies attempted to introduce the CO₂ concentrating mechanisms of photosynthetic bacteria or C4 plants into C3 plants, so far with little or no success.

For example, studies attempting to genetically modify RubisCO in order to raise its affinity for CO₂ [35] and transformation of a C3 plant (rice) with several genes responsible for C4 metabolism have been described [36-40].

Although theoretically such approaches can lead to enhanced CO₂ fixation in C3 plants, results obtained from such studies have been disappointing.

There is thus a widely recognized need for, and it would be highly advantageous to have, a method of generating plants and crops exhibiting enhanced growth and/or increased commercial yields.

### SUMMARY OF THE INVENTION

According to one aspect of the present invention there is provided a method of obtaining plants characterized by enhanced growth and/or commercial yield under growth limiting conditions, the method comprising the steps of: a) obtaining a population of plants transformed to express a polypeptide having an amino acid sequence at least 60% homologous to that set forth in SEQ ID NOs: 3, 5, 6, 7, 10, 11, 12 or 13; b) growing said population of plants under the growth limiting condition to thereby detect plants of said population having enhanced growth and/or commercial yield; and c) selecting plants expressing said polypeptide having enhanced growth and/or commercial yield as compared to control plants, thereby obtaining plants characterized by enhanced growth and/or commercial yield under growth limiting conditions.

According to further features in the described preferred embodiments step (a) is effected by transforming at least a portion of the plants of said population with a nucleic acid construct comprising a polynucleotide region encoding said polypeptide.

According to still further features in the described preferred embodiments the transforming is effected by a method selected from the group consisting of Agrobacterium mediated transformation, viral infection, electroporation and particle bombardment.

According to yet further features in the described preferred embodiments the nucleic acid construct further comprises a second polynucleotide region encoding a transit peptide, the second polynucleotide being operably linked to the polynucleotide region encoding the polypeptide having an amino acid sequence at least 60% homologous to that set forth in SEQ ID NOs: 3, 5, 6, 7, 10, 11, 12 or 13.

According to still further features in the described preferred embodiments the nucleic acid construct further comprises a promoter sequence operably linked to said polynucleotide region encoding said polypeptide having an amino acid sequence at least 60% homologous to that set forth in SEQ ID NOs: 3, 5, 6, 7, 10, 11, 12 or 13.

According to further features in the described preferred embodiments the nucleic acid construct further comprises a promoter sequence operably linked to both said polynucleotide region encoding said polypeptide having an amino acid sequence at least 60% homologous to that set forth in SEQ ID NOs: 3, 5, 6, 7, 10, 11, 12 or 13 and to said second polynucleotide region.

According to still further features in the described preferred embodiments the promoter is functional in eukaryotic cells.

According to still further features in the described preferred embodiments the promoter is selected from the group consisting of a constitutive promoter, an inducible promoter, a developmentally regulated promoter and a tissue specific promoter.

According to another aspect of the present invention there is provided a transformed crop comprising a population of transformed plants expressing a polypeptide having an amino acid sequence at least 60% homologous to that set forth in SEQ ID NOs: 3, 5, 6, 7, 10, 11, 12 or 13 wherein each individual plant of said population is characterized by enhanced growth under limiting conditions as compared to similar non transformed plants when grown under at least one growth limiting condition.

According to further features in the described preferred embodiments the amino acid sequence is as set forth by SEQ ID NOs: 3, 5, 6, 7, 10, 11, 12 or 13.

According to yet further features in preferred embodiments of the invention described below, the plants are grown in an environment characterized by at least one growth limiting condition selected from the group consisting of water stress, low humidity, salt stress, and/or low CO₂ conditions.

According to still further features in the described preferred embodiments the plant is grown in an environment characterized by a CO₂ concentration similar to or lower than in air, (approximately 0.035% CO₂ in air, and 10 micromolar CO₂ in solution) and/or humidity lower than 40%.

According to still further features in the described preferred embodiments the plants are C3 plants.

According to still further features in the described preferred embodiments the C3 plants are selected from the group consisting of tomato, soybean, potato, cucumber, cotton, wheat, rice, barley, sunflower, banana, tobacco, lettuce, cabbage, petunia, solidago and poplar.

According to still further features in the described preferred embodiments the plants are C4 plants.

According to still further features in the described preferred embodiments the C4 plants are selected from the group consisting of corn, sugar cane and sorghum.

According to still further features in the described preferred embodiments a growth rate of the population of transformed plants is at least 10% higher than that of a population of similar non transformed plants when both are grown under a similar growth limiting condition.

According to still further features in the described preferred embodiments the growth rate is determined by at least one growth parameter selected from the group consisting of increased fresh weight, increased dry weight, increased root growth, increased shoot growth and flower development over time.

According to still further features in the described preferred embodiments the transformed plant is further characterized by an increased commercial yield as compared to similar non transformed plants grown under similar conditions.

According to yet another aspect of the present invention there is provided a nucleic acid expression construct comprising: (a) a first polynucleotide region encoding a polypeptide including an amino acid sequence at least 60% homologous to that set forth by SEQ ID NOs: 3, 5, 6, 7, 10, 11, 12 or 13; and (b) a second polynucleotide region comprising a promoter sequence operably linked to said first polynucleotide region, the promoter sequence being functional in eukaryotic cells.

According to still further features in the described preferred embodiments the promoter is selected from the group consisting of a constitutive promoter, an inducible promoter and a tissue specific promoter.

According to still further features in the described preferred embodiments the promoter is a plant promoter.

According to still further features in the described preferred embodiments the nucleic acid expression construct further comprises a second polynucleotide region encoding a transit peptide, the second polynucleotide being operably linked to the polynucleotide region encoding the polypeptide having an amino acid sequence at least 60% homologous to that set forth in SEQ ID NOs: 3, 5, 6, 7, 10, 11, 12 or 13.

The present invention successfully addresses the shortcomings of the presently known configurations by providing plants and crops characterized by enhanced growth and to methods and nucleic acid constructs useful for generating same.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic representation of a genomic region in Synechococcus sp. PCC 7942 where an insertion (indicated by a star) of an inactivation library fragment led to the formation of mutant IL-2. DNA sequence is available in the GenBank, Accession number U62616. Restriction sites are marked as: A—ApaI, B—BamHI, Ei—EcoRI, E—EcoRV, H—HincII, Hi—HindIII, K—KpnI, M—MfeI, N—NheI, T—TaqI. Underlined letters represent the terminate position of the DNA fragments that were used as probes. Relevant fragments isolated from an EMBL3 library are marked E1, E2 and E3. P1 and P2 are fragments obtained by PCR. Triangles indicate sites where a cartridge encoding Kan^{r} was inserted. Open reading frames are marked by an arrow and their similarities to other proteins are noted. Sll and slr (followed by four digits) are the homologous genes in Synechocystis sp. PCC 6803 [23]; YZ02-myctu, Accession No. Q10536; ICC, Accession No. P36650; Y128-SYNP6, Accession No. P05677; YGGH, Accession No. P44648; Ribosome binding factor A homologous to sll0754 and to P45141; O-acetylhomoserine sulfhydrylase homologous to sll10077 and NifS. ORF280 started upstream of the schematic representation presented herein.

FIG. 2 shows nucleic acid sequence alignment between ORF467 (ICTB, SEQ ID NO:2) and slr1515 (SLR, SEQ ID NO:4). Vertical lines indicate nucleotide identity. Gaps are indicated by hyphens. Alignment was performed using the Blast software where gap penalty equals 10 for existence and 10 for extension, average match equals 10 and average mismatch equals −5. Identical nucleotides equals 56%.

FIG. 3 shows amino acid sequence alignment between the IctB protein (ICTB, SEQ ID NO:3) and the protein encoded by slr1515 (SLR, SEQ ID NO:5). Identical amino acids are marked by their single letter code between the aligned sequences, similar amino acids are indicated by a plus sign. Alignment was performed using the Blast software where gap open penalty equals 11, gap extension penalty equals 1 and matrix is blosum62. Identical amino acids equals 47%, similar amino acids equals 16%, total homology equals 63%.

FIGS. 4a-b are graphs showing the rates of CO₂ and of HCO₃⁻ uptake by Synechococcus PCC 7942 (4a) and mutant IL-2 (4b) as a function of external Ci concentration. LC and HC are cells grown under low (air) or high CO₂ (5% CO₂ in air), respectively. The rates were assessed from measurements during steady state photosynthesis using a membrane inlet mass spectrometer (MIMS) [6, 7, 22].

FIG. 5 presents DNA sequence homology comparison of a region of ictB found in Synechococcus PCC 7942 and in mutant IL-2. This region was duplicated in the mutant due to a single cross-over event. Compared with the wild type, one additional nucleotide and a deletion of six nucleotides were found in the BamHI side, and 4 nucleotides were deleted in the ApaI side (see FIG. 1). These changes resulted in stop codons in IctB after 168 or 80 amino acids in the BamHI and ApaI sides, respectively. The sequence shown by this Figure starts from amino acid 69 of ictB.

FIG. 6 illustrates the ictB construct used in generating the transgenic plants of the present invention, including a 35S promoter, the transit peptide (TP) from the small subunit of pea Rubisco (nucleotide coordinates 329-498 of GeneBank Accession number x04334 where we replaced the G in position 498 with a T, the ictB coding region, the NOS termination and kanamycin-resistance (Kn^{R}) within the binary vector pBI121 from Clontech.

FIG. 7 is a Northern blot analysis of transgenic and wild type (w) Arabidopsis and tobacco plants using both ictB and 18S rDNA as probes.

FIG. 8 illustrates the rate of photosynthesis as affected by the intercellular concentration of CO₂ in wild type and the transgenic tobacco plants of the present invention; plants 1 and 11 are transgenic.

FIG. 9 illustrates growth experiments conducted on both transgenic (A, B and C) and wild type (WT) Arabidopsis plants. Each growth pot included one wild type and three transgenic plants. Data are provided as the average dry weight of the plants ±S.D. Growth conditions are described in the Examples section.

FIGS. 10a-b are hydropathy plots of the IctB protein from Synechococcus PCC 7942 and homologous protein Synwh0268 from Synechococcus sp. Strain WH 8102. Note the 10 clearly identified transmembrane (highly hydrophobic) and several hydrophilic domains common to both proteins. Analysis was performed using TopPred program (http://bioweb.pasteur.fr/cgi-bin/seqanal/toppred.pl).

FIG. 11 shows the alignment of ictB amino acid sequence with sequences from homologous proteins of several cyanobacteria. The alignment was performed using the CLUSTALW multiple alignment program. Note the highly conserved hydrophilic region (position 308-375) having strong homology (46.3% identity and 20.9% similarity) between the proteins from different cyanobacteria. Red indicates identity (star), green strong similarity (colon) and blue similarity (dot).

FIG. 12 is a graphic demonstration of enhanced inorganic carbon fixation under low humidity by transgenic tobacco plants expressing the ictB gene. RubisCO activity is expressed as rate of carboxylation, measured in nmol CO₂ fixed per nmol active sites per minute. Note the clear advantage of the transgenic plants (open circle) over the wild type (open square) under limiting CO₂ conditions (in-vivo). Rate of carboxylation is expressed in nmol CO₂ fixed per nmol active sites per minute. Inset is a graphic representation of the kinetics of carboxylation, expressed as S/V vs. S, for transgenic and wild type tobacco plants. Note the higher reaction rate (Vmax) but similar substrate affinity (Km) of the carboxylation reaction in the transgenic plants.

### DESCRIPTION OF THE PREFERRED EMBODIMENTS

The present invention is of a method of generating plants characterized by enhanced growth and/or fruit yield and/or flowering rate, of plants generated thereby and of nucleic acid constructs utilized by such a method. Specifically, the present invention can be used to substantially increase the growth rate and/or fruit yield of C3 plants especially when grown under growth-limiting conditions characterized by low humidity and/or a low CO₂ concentration.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Increasing the growth size/rate and/or commercial yield of crop plants is of paramount importance especially in regions in which growth/cultivation conditions are suboptimal due to a lack of, for example, water.

While reducing the present invention to practice the inventors have discovered that plants expressing exogenous polynucleotides encoding a putative cyanobacterial inorganic carbon transporter are characterized by enhanced growth, especially when grown under growth limiting conditions characterized by low humidity or low CO₂ concentrations.

Thus, according to the present invention there is provided a transformed plant expressing a polypeptide including an amino acid sequence which is at least 60% homologous to that set forth in SEQ ID NO: 3, 5, 6, 7, 10, 11, 12 or 13.

As is further described hereinbelow, the transformed plant of the present invention is characterized by enhanced growth as compared to similar non transformed plants grown under similar growth conditions, and thus can be identified and selected for by exposing plants expressing the polypeptide sequence of the present invention to growth limiting conditions.

As used herein, the phrase “enhanced growth” refers to an enhanced growth rate, or to an increased growth size/weight of the whole plant or preferably the commercial portion of the plant (increased yield) as determined by fresh weight, dry weight or size of the plant or commercial portion thereof.

As is further detailed in the Examples section which follows, the transformed plants of the present invention exhibit, for example, a growth rate which is at least 10% higher than that of a similar non transformed plant when both plants are grown under similar growth limiting conditions.

According to a preferred embodiment of the present invention, the polypeptide is at least 60%, preferably at least 65%, more preferably at least 70%, still more preferably at least 75%, yet more preferably at least 80%, more preferably at least 85%, more preferably at least 90%, yet more preferably at least 95%, ideally 95-100% homologous (identical+similar) to SEQ ID NO: 3, 5, 6, 7, 10, 11, 12 or 13 or a portion thereof as determined using the Blast software where gap open penalty equals 11, gap extension penalty equals 1 and matrix is blosum62.

According to preferred embodiments of the present invention, the growth limiting conditions are characterized by humidity of less than 40% and/or CO₂ concentration which is lower than in air.

The transformed plant of the present invention can be any plant including, but not limited to, C3 plants such as, for example, tomato, soybean, potato, cucumber, cotton, wheat, rice, barley or C4 plants, such as, for example, corn, sugar cane, sorghum and others.

The transformed plants of the present invention are generated by introducing a nucleic acid molecule or polynucleotide encoding the polypeptide(s) described above into cells of the plant.

Such a nucleic acid molecule or polynucleotide can have a sequence corresponding to at least a portion of SEQ ID NO:2, 4, 8 or 9, the portion encoding a polypeptide contributing the increased growth trait.

Alternatively or additionally the nucleic acid molecule can have a sequence which is at least 60%, preferably at least 65%, more preferably at least 70%, still more preferably at least 75%, yet more preferably at least 80%, more preferably at least 85%, more preferably at least 90%, yet more preferably at least 95%, ideally 95-100% identical to that portion, as determined using the Blast software where gap penalty equals 10 for existence and 10 for extension, average match equals 10 and average mismatch equals −5. It will be appreciated in this respect that SEQ ID NO:2, 4, 8 or 9 can be readily used to isolate homologous sequences which can be tested as described in the Examples section that follows for their bicarbonate transport activity. Methods for isolating such homologous sequences are extensively described in, for example, Sambrook et al. [9] and may include hybridization and PCR amplification.

Still alternatively or additionally the nucleic acid molecule can have a sequence capable of hybridizing with the portion of SEQ ID NO:2, 4, 8 or 9. Hybridization for long nucleic acids (e.g., above 200 bp in length) is effected according to preferred embodiments of the present invention by stringent or moderate hybridization, wherein stringent hybridization is effected by a hybridization solution containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C.; whereas moderate hybridization is effected by a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

Preferably, the polypeptide encoded by the nucleic acid molecule of the present invention includes an N terminal transit peptide fused thereto which serves for directing the polypeptide to a specific membrane. Such a membrane can be, for example, the cell membrane, wherein the polypeptide will serve to transport bicarbonate from the apoplast into the cytoplasm, or, such a membrane can be the outer and preferably the inner chloroplast membrane. Transit peptides which function as herein described are well known in the art. Further description of such transit peptides is found in, for example, Johnson et al. The Plant Cell (1990) 2:525-532; Sauer et al. EMBO J. (1990) 9:3045-3050; Mueckler et al. Science (1985) 229:941-945; Von Heijne, Eur. J. Biochem. (1983) 133:17-21; Yon Heijne, J. Mol. Biol. (1986) 189:239-242; Iturriaga et al. The Plant Cell (1989) 1:381-390; McKnight et al., Nucl. Acid Res. (1990) 18:4939-4943; Matsuoka and Nakamura, Proc. Natl. Acad. Sci. USA (1991) 88:834-838. A recent text book entitled “Recombinant proteins from plants”, Eds. C. Cunningham and A. J. R. Porter, 1998 Humana Press Totowa, N.J. describe methods for the production of recombinant proteins in plants and methods for targeting the proteins to different compartments in the plant cell. The book by Cunningham and Porter is incorporated herein by reference. It will however be appreciated by one of skills in the art that a large number of membrane integrated proteins fail to posess a removable transit peptide. It is accepted that in such cases a certain amino acid sequence in said proteins serves not only as a structural portion of the protein, but also as a transit peptide.

Preferably, the nucleic acid molecule of the present invention is included within a nucleic acid construct designed as a vector for transforming plant cells thereby enabling expression of the nucleic acid molecule within such cells.

Plant expression can be effected by introducing the nucleic acid molecule of the present invention (preferably using the nucleic acid construct) downstream of a plant promoter present in endogenous genomic or organelle polynucleotide sequences (e.g., chloroplast or mitochondria), thereby enabling expression thereof within the plant cells.

In such cases, the nucleic acid construct further includes sequences which enable to “knock-in” the nucleic acid molecule into specific or random polynucleotide regions of such genomic or organelle polynucleotide sequences.

Preferably, the nucleic acid construct of the present invention further includes a plant promoter which serves for directing expression of the nucleic acid molecule within plant cells.

As used herein in the specification and in the claims section that follows the phrase “plant promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric.

Thus, the plant promoter employed can be a constitutive promoter, a tissue specific promoter, an inducible promoter or a chimeric promoter.

Examples of constitutive plant promoters include, without limitation, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PHSβ promoter, zein storage protein promoter, conglutin gamma promoter from soybean, AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

The nucleic acid construct of the present invention preferably further includes additional polynucleotide regions which provide a broad host range prokaryote replication origin; a prokaryote selectable marker; and, for Agrobacterium transformations, T DNA sequences for Agrobacterium-mediated transfer to plant chromosomes. Where the heterologous sequence is not readily amenable to detection, the construct will preferably also have a selectable marker gene suitable for determining if a plant cell has been transformed. A general review of suitable markers for the members of the grass family is found in Wilmink and Dons, Plant Mol. Biol. Reptr. (1993) 11:165-185.

Suitable prokaryote selectable markers include resistance toward antibiotics such as ampicillin, kanamycin or tetracycline. Other DNA sequences encoding additional functions may also be present in the vector, as is known in the art.

Sequences suitable for permitting integration of the heterologous sequence into the plant genome are also recommended. These might include transposon sequences as well as Ti sequences which permit random insertion of a heterologous expression cassette into a plant genome.

The nucleic acid construct of the present invention can be utilized to stably or transiently transform plant cells. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of effecting stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Amtzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

Additional methods of transgenic plant propagation and transformation are described in U.S. Pat. Nos. 6,610,909 to Oglevee-O'Donavan et al, and 6,384,301 to Martinell et al, both incorporated herein by reference.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid; and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

While reducing the present invention to practice, transgenic Arabidopsis and tobacco plants expressing the ictB polypeptide characterized by enhanced growth, photosynthesis and inorganic carbon fixation were generated. It will be appreciated that within a population of plants transformed to express the ictB polypeptide, or homologous polypeptide sequences associated with inorganic carbon uptake, plants having enhanced photosynthesis and inorganic carbon fixation, may not all be characterized by enhanced growth, since plant growth is a complex process dependent on a multitude of factors, of which rate of photosynthesis and inorganic carbon fixation are but two. Some of the other crucial factors for plant growth are levels of plant hormones such as brassinosteroids and cytokinins (see Yin et al, PNAS USA 2002;99:10191-96, and Werner et al, PNAS USA 2001;98:10487-92), nitrogen availability (Fritschi et al Agron Jour 2003;95:133-46) and mineral availability (Brauer et al Crop Sci 2002;42:1640-46). Improvement of plant growth parameters, such as dry weight and biomass, requires careful coordination of these many factors. An increase or decrease in one or the other does not necessitate comparable effects on the overall process of growth.

Indeed, it has been demonstrated that increased photosynthesis, measured in isolation, does not necessarily lead to enhanced growth. In one example, Makino et al (J Exp Bot. 2000;51:383-89) produced transgenic plants having up to 15% increased photosynthesis as compared to wild type, but no greater biomass production. Similarly, increased crop yields can be achieved without improving photosynthesis rate, as has been demonstrated by the semi-dwarf “green revolution” rice, in which a deficiency in plant growth hormones (GA) paradoxically produced record increases in rice yields throughout Asia (see, for example, Speilmeyer et al, PNAS USA 2002;99: 9043-8). Thus, transformed plants characterized by enhanced growth need to be identified and isolated from among the transformed plant population, by applying suitable selection criteria so as to distinguish such plants for further propagation.

Such selection criteria suitable for use with the methods and populations of transformed plants of the present invention are described in detail in the Examples section which follows hereinbelow. Typically, plants transformed to express the ictB polypeptide, or homologous polypeptide sequences associated with inorganic carbon uptake are exposed to growth limiting conditions comprising water stress, low humidity, salt stress, and/or low CO₂ conditions. Preferably, these conditions comprise humidity lower than 40% and/or an intercellular CO₂ concentration lower than 10 micromolar. Exposure to such conditions may be effected in field conditions or in controlled, isolated environments such as climate controlled greenhouses or growth chambers.

Following exposure to such growth limiting conditions, for example, at predetermined intervals of hours, days, months or more, growth of the transformed plants can be assessed, and plants having enhanced growth under limiting conditions identified and selected using a variety of growth parameters familiar to one of ordinary skill in the art. Suitable growth parameters, and methods for their assessment are described in detail in the Examples section hereinbelow. Preferred growth parameters include fresh weight, dry weight, enhanced biomass, root growth, shoot growth and flower development. Biomass may be root biomass, vegetative organ biomass, and/or whole plant biomass. Methods for detection of enhanced biomass and other growth parameters are disclosed herein, and widely known and practiced (see, for example, U.S. Pat. No. 6,559,357 to Fischer et al). Selected plants which have a polynucleotide encoding ictB stably integrated into their genome, and exhibiting enhanced growth, can be repropagated and cultivated, and the resultant populations of stably transformed plants subjected to additional cycle(s) of exposure to growth limiting conditions and selection, producing plant populations and/or crops wherein each individual plant of said population is characterized by enhanced growth under limiting conditions as compared to similar non transformed plants when grown under a growth limiting condition.

Repropagation of selected plants having ictB expression and exhibiting enhanced growth can be effected by any of the well known methods of plant regeneration (see, for example, the methods described hereinabove, and methods of selfing and seed propagation described in U.S. Pat. No. 6,414,223 to Kodali, et al, which is incorporated herein by reference). In one preferred embodiment repropagation is effected by growing the selected plants to seed, collecting mature seeds from the selected plants, planting the seeds and cultivating the resultant plants under limiting conditions, thereby producing a second population of plants having ictB expression and characterized by enhanced growth under limiting conditions. As described hereinabove, the resultant populations of stably transformed plants can be subjected to repeated continuous or intermittent cycles of selection, recultivation and seed collection in order to producing plant populations and/or crops wherein each individual plant of said population is characterized by enhanced growth under limiting conditions as compared to similar non transformed plants when grown under a growth limiting condition.

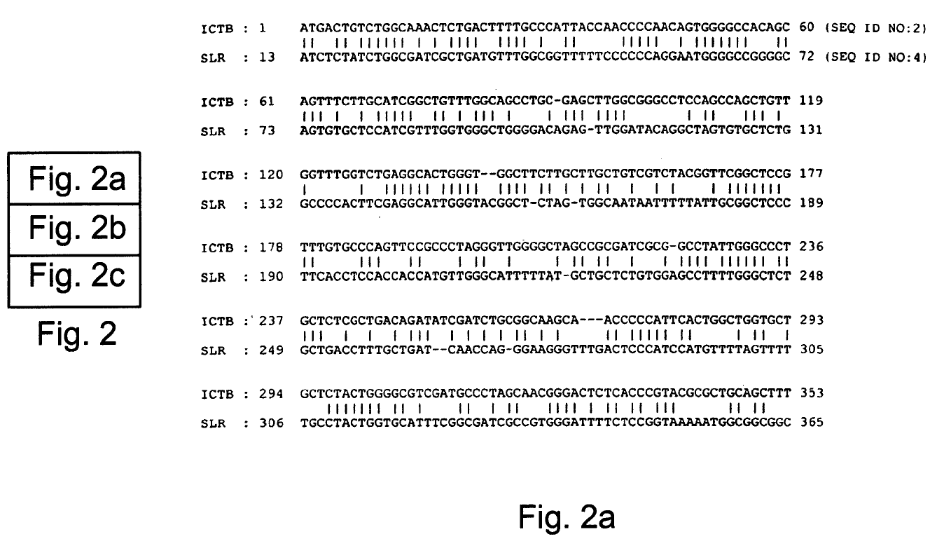

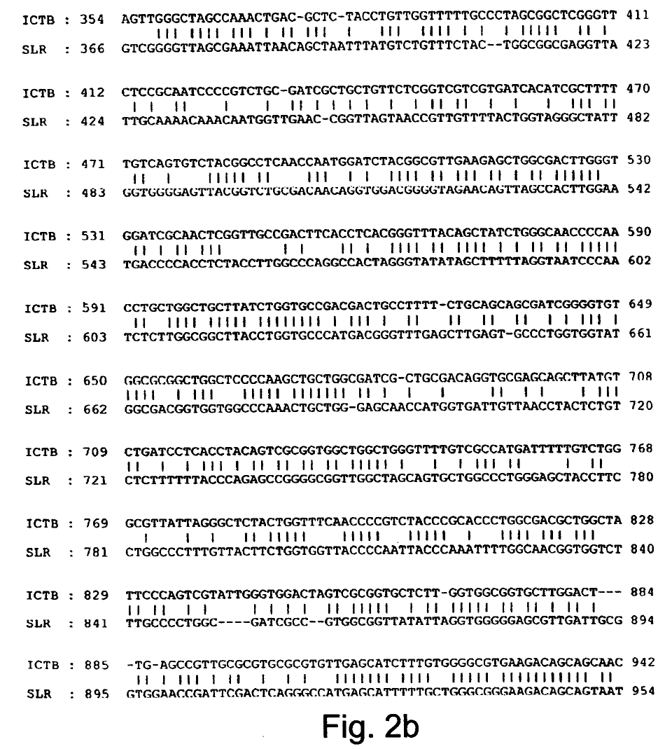

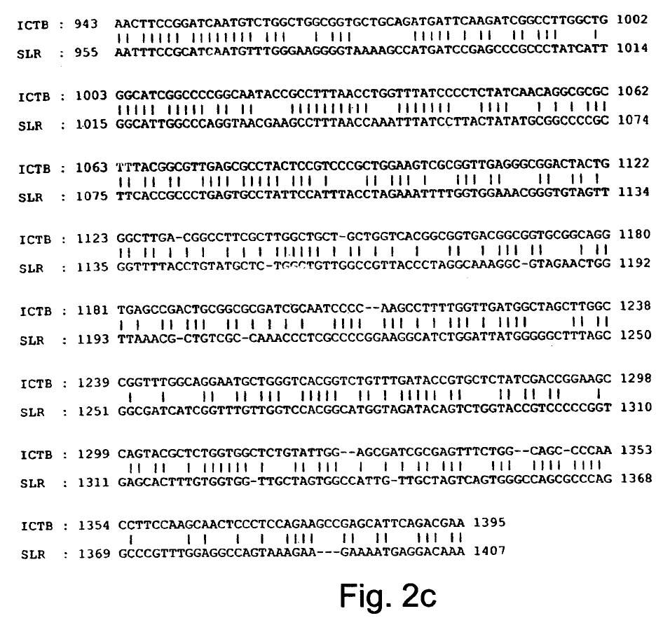

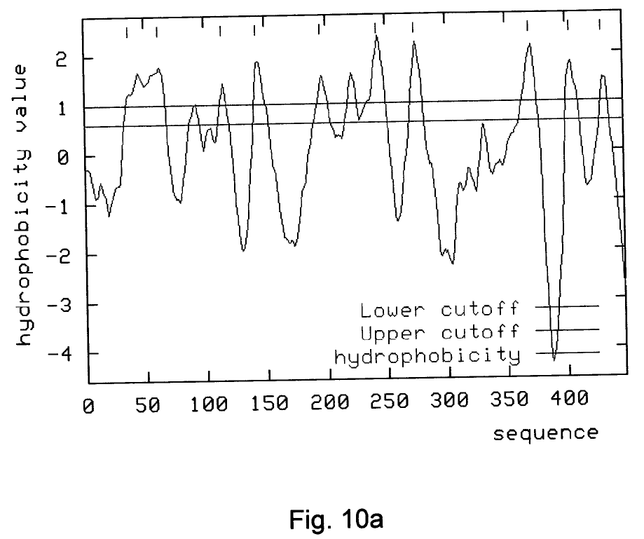

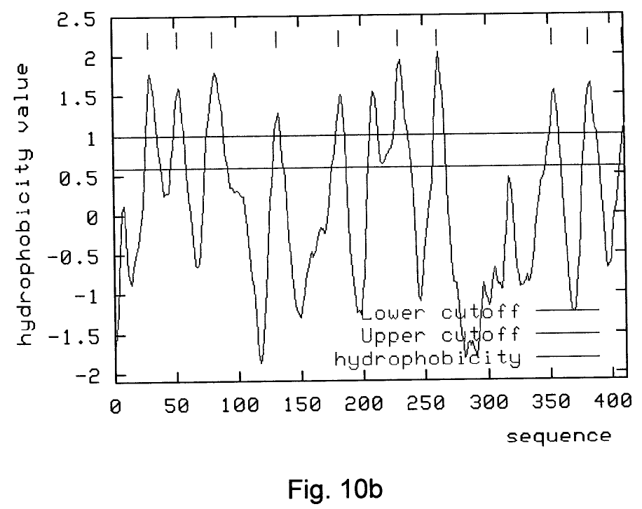

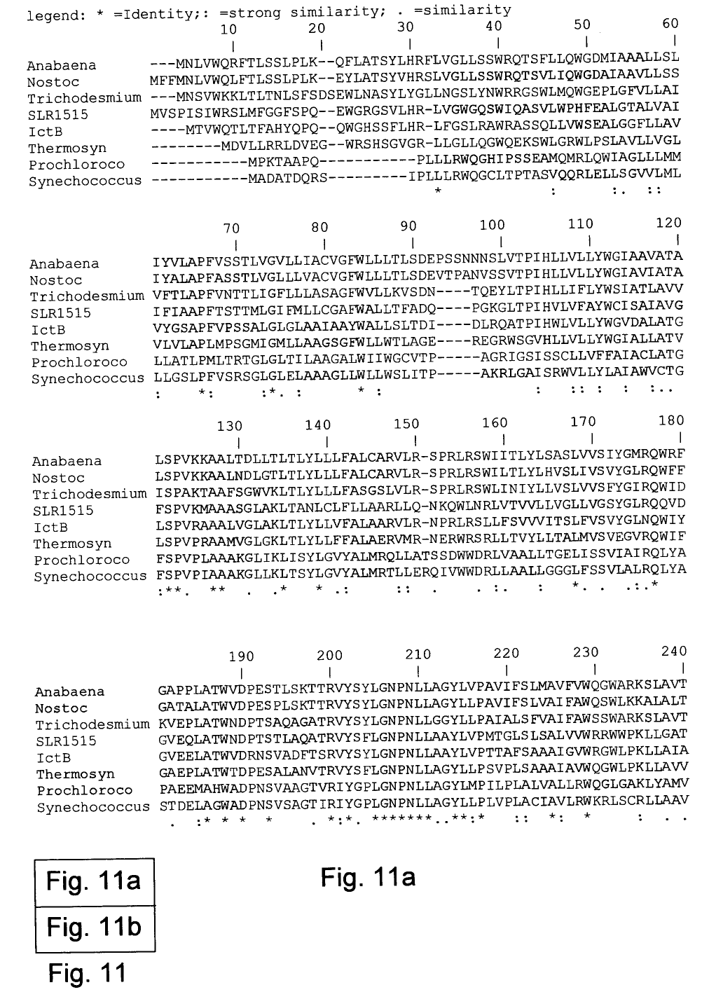

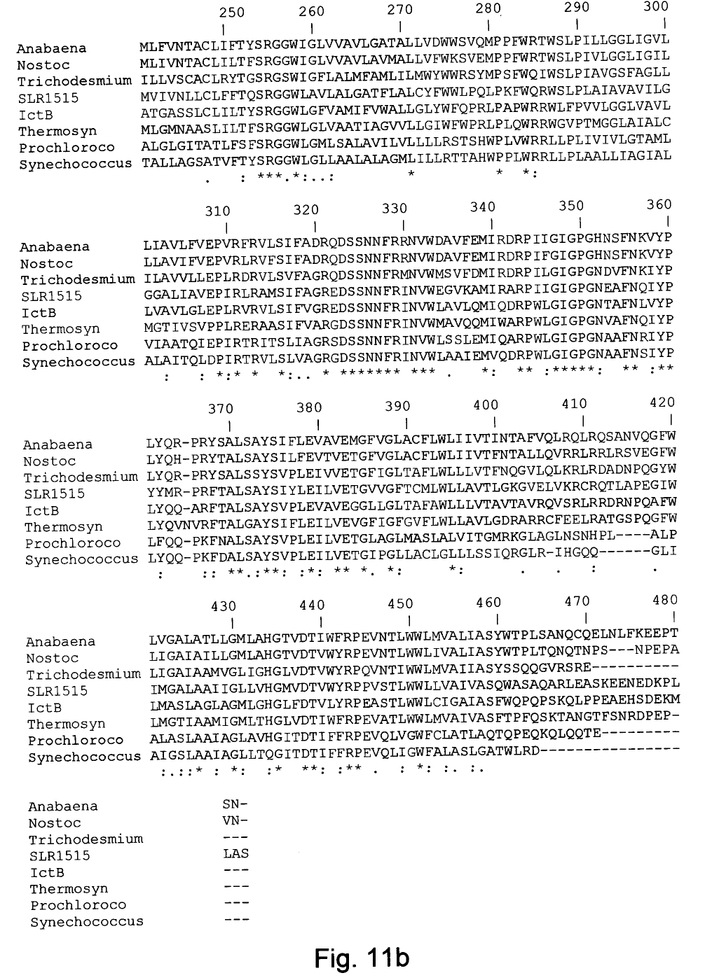

While reducing the present invention to practice, it was found that all published genomes of photosynthetic cyanobacteria have sequences highly homologous to that of the ictB coding sequence (SEQ. ID. NO:2) (FIG. 11). Further, it has been demonstrated that the site of inactivation in the transposon-inactivated mutant in the cyanobacterium Synechocystis PCC 6803, is a gene having a high level of homology to the ictB sequence from the IL-2 mutant of Synechococcus PCC 7942 (see slr1515 in FIGS. 2 and 11, and Bonfil et al., FEBS Letters 1998;430:236-40). Sequence comparison of cyanobacteria polypeptide sequences homologous to ictB reveals that the transmembrane domains, and the long hydrophilic domain are highly conserved in all members of this family (FIGS. 10a and b, and 11). Such a configuration of 10 transmembrane domains is also found in the RBC band 3 bicarbonate transporter protein from humans, and is characteristic of many transporter proteins.

Thus, the sequences of present invention may be used for identification and isolation of sequences of other species coding for homologous polypeptides associated with inorganic carbon transport, capable of enhancing photosynthesis and growth under growth limiting conditions. Sequences coding for such functional equivalents of the ictB polypeptide, such as the homologous sequences shown in FIG. 11, can also be used for the generation of transgenic plants having enhancing photosynthesis and growth under growth limiting conditions by transformation, expression and selection according to the methods of the present invention.

There are a number of well known molecular techniques that can be used successfully by one of ordinary skill in the art to generate a range of homologous function equivalents of the ictB polypeptide from divergent species having low CO₂ acclimation capability.

Using such methods, one of ordinary skill in the art privileged to the teachings of the present invention would easily be capable of isolating mRNAs, synthesizing cDNA (or screening cDNA libraries) and generating constructs suitable for cloning and expressing sequences homologous to ictB. Similarly the teachings of the present invention could just as easily be used to guide the ordinary artisan in isolating and cloning appropriate genomic sequences.

It will be appreciated that the isolation of a gene, or a number of genes encoding sequences homologous to, and having equivalent biological function to a defined sequence, constituting a family of functional equivalents, is a well known, art recognized technique. One of ordinary skill in the art may employ any of a number of well-known approaches highly suitable for screening for homologous genes, such as:

Homology screening: Once an interesting gene has been isolated from one species (i.e., ictB from Synechococcus in this case) it is well within the ability of one of an ordinary skill in the art to use moderately high stringency hybridization conditions to isolate cDNAs from other species. Likewise additional family members from the same species can be similarly identified. Examples of homology screening and moderately high stringency hybridization conditions are well known (see details hereinabove and, for example, U.S. Pat No. 6,391,550, to Lockhart et al. and U.S. Pat. No. 6,232,061 to Marchionni et al);

PCR-based screening with specific PCR primers designed and used to amplify homologous regions of DNA or reverse transcriptase products of mRNAs of a given tissue, cell or cell compartment, and screening of cDNA libraries with the amplification products. Reverse transcriptase can be used to extend a primer, which has been designed to anneal to a conserved sequence. It will be appreciated that such products can be heterogeneous since different reverse transcriptase molecules would extend to different degrees. To produce a fragment of a unique size, restriction enzymes capable of cleaving single stranded DNA can be used. Once a fragment is obtained it is homopolymer-tailed using terminal transferase. The tailored sequence can then be used as a site to anchor a complementary oligonucleotide sequence. If the primer is extended the resulting product will be suitable for PCR amplification between the two primers which were used in its synthesis;

Differential display—This approach of isolating homologous DNA sequences relies not on knowledge of their primary sequences, rather on assumptions about their expression. In this method spatially and/or temporally differentially expressed genes are identified. For example, as disclosed in the instant invention, it is conceivable that due to their protective disposition, polypeptides of the bicarbonate transporter family will be expressed under conditions of low Ci availability. Briefly, mRNA is isolated from two populations of cells exposed to divergent conditions, and reverse transcribed to produce two representative populations of cDNAs. Aliquots of these cDNAs can then be converted to probes by random hexamer priming and used to screen duplicate lifts from a target library (such as a membrane library). Any plaque or colony, for which to one probe but not the other hybridizes to duplicate lifts from a library, is a potential candidate of interest. Differential expression can be tested by Northern analysis or a related approach.

Database screening—The rapid accumulation of sequence information and genetic data allows the elimination of steps required to isolate cDNAs. By employing global or local alignment algorithms, homologous sequences of a cDNA of interest (i.e., ictB) may be identified.

Given the low homology of the ictB polypeptide sequence to other, unrelated sequences, and the highly conserved homology among similar sequences from other cyanobacteria species (see FIG. 11), it is highly likely that any sequence identified according to the teachings of the present invention, described hereinabove, will constitute a putative member of the newly identified family of inorganic carbon transporters. Gene Family Isolation Services have recently become commercially available (see, for example, Resgene “Gene and Gene Family Isolation Services”, cat # SGT 1001, Invitrogen Corp; Cellular and Molecular Technologies, Inc at www.cmt.com; Pangene Corporation, Freemont Calif.; and Homologous Cloning Service of Evrogene JSC, Moscow, Russia), further simplifying identification and isolation of homologous gene families. Further validation of putative homologous sequences can be effected according to selection criteria of biological activity, molecular weight, cellular localization, immune reactivity, etc. Thus, one of ordinary skill in the art privileged to the teachings of the present invention would be capable of isolating mRNAs, or screening cDNA libraries to identify and generate constructs representing expressed sequences homologous to the polynucleotide sequence of the present invention. Techniques for isolation of such homologous gene families by “Homology Cloning” are well known in the art (see, for example, U.S. Pat No. 6,391,550, to Lockhart et al. and U.S. Pat. No. 6,232,061 to Marchionni et al).

The methods of the present invention provide guidelines which can be used to test functional characteristics of expressed polypeptides homologous to ictB:

(i) Directed mutation assays—mutation in the homologous gene can be introduced by well known molecular techniques, and the operation of the CO₂ concentrating mechanism assayed. Impairment of growth under conditions of low CO₂ concentration, as described in the Examples section hereinbelow, would indicate a CO₂ concentrating function of the homologous gene.

(ii) Function in transgenic plants—Members of the family of ictB homologues can be cloned and expressed in diverse plant hosts according to the methods and techniques described in herein (see above, and the Examples section hereinbelow), transformants selected, and assessed for enhanced photosynthesis, reduction in compensation point, enhanced RubisCO activity, and enhanced growth, as detailed in the Examples section hereinbelow. Thus, members of the family of ictB functional homologues having photosynthesis, inorganic carbon fixation and growth enhancing activity can be used in the generation of plants and crops having enhanced growth under growth limiting conditions, according to the methods of the present invention. Further validation of putative homologous sequences can be effected according to selection criteria such as molecular weight and antibody reactivity.

In one embodiment, functional homologues of the ictB are polypeptides having at least 60%, preferably 70%, more preferably 80%, most preferably 90%, and ideally 95-100% homology to the polypeptide set forth in SEQ ID NO:3, having photosynthesis, inorganic carbon fixation and growth enhancing activity when expressed in plants. Similarly, polynucleotides encoding such functional homologues, identified and isolated using the methods described herein, can be used for generating plants having enhanced growth according to the methods of the present invention.

It will be appreciated, in the context of the present invention, that polypeptides which share 60% homology or more are essentially the same functional polypeptide including contiguous or non-contiguous functional variants thereof (see For example U.S. Pat. Nos: 6,342,583, 6,352,832 and 6,331,284). Families of polypeptides having similar catalytic activity, such as the Alcohol Dehydrogenase (ADH) family (see: Deuster, G Eur J Biochem 2000;267:4315-4328) and the cytochrome c1 family (see cytochrome c1 at www.ExPASy.org, niceprot) maintain substantial amino acid homology of 60% or greater even between unrelated species. A functional equivalent (i.e., homologue) refers to a polypeptide, which does not have the exact same amino acid sequence of ictB (SEQ ID NO:3) due to deletions, mutations or additions of one or more contiguous or non-contiguous amino acid residues but retains biological activity of the naturally occurring polypeptide (i.e., enhanced inorganic carbon fixation). The functional equivalent can have conservative changes wherein a substituted amino acid has similar structural or chemical properties. More rarely, a functional equivalent has non-conservative changes e.g., replacement of glycine with tryptophan. Similar minor variations can also include amino acid deletions, insertions or both.

Guidance in determining which and how many amino acids may be substituted, inserted or deleted without abolishing biological or immunological activity can be found in the specifications (further summarized hereinunder) and using computer programs well known in the art, such as, DNAStar software (DNAStar Inc. http://www.dnastar.com/default.html), which utilizes known algorithms. For example, amino acid substitutions may be made on the basis of similarity, polarity, charge, solubility, hydrophilicity and/or amphipathic nature of the residues, as long as the disclosed biological activity is retained. Based upon these considerations, arginine, lysine and histidine; alanine, glycine and serine; and phenylalanine, tryptophan and tyrosine; are defined in the art as examples of biologically functional equivalents (see U.S. Pat. Nos: 4,554,101 and 6,331,284).

Thus, the present invention provides methods, nucleic acid constructs and transformed plants and crops generated using such methods and constructs, which transformed plants are characterized by an enhanced growth rate and/or increased commercial yield.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### ictB Isolation and Characterization

### Materials and Experimental Methods

Growth Conditions:

Cultures of Synechococcus sp. strain PCC 7942 and mutant IL-2 thereof were grown at 30° C. in BG₁₁ medium supplemented with 20 mM Hepes-NaOH pH 7.8 and 25 μg mL⁻¹ kanamycin (in the case of the mutant). The medium was aerated with either 5% v/v CO₂ in air (high CO₂) or 0.0175% v/v CO₂ in air (low CO₂) which was prepared by mixing air with CO₂-free air at a 1:1 ratio. Escherichia coli (strain DH5α) were grown on an LB medium [9] supplemented with either kanamycin (50 μg/mL) or ampicillin (50 μg/mL) when required.

Measurements of Photosynthesis and Ci Uptake:

The rates of inorganic carbon (Ci)-dependent O₂ evolution were measured by an O₀₂ electrode as described elsewhere [10] and by a membrane inlet mass spectrometer (MIMS, [6, 11]). The MIMS was also used for assessments of CO₂ and HCO₃⁻ uptake during steady state photosynthesis [6]. Ci fluxes following supply of CO₂ or HCO₃⁻ were determined by the filtering centrifugation technique [10]. High-CO₂ grown cells in the log phase of growth were transferred to either low or high CO₂ 12 hours before conducting the experiments. Following harvest, the cells were resuspended in 25 mM Hepes-NaOH pH 8.0 and aerated with air (Ci concentration was about 0.4 mM) under light flux of 100 μmol photon quanta m⁻² s⁻¹. Aliquots were withdrawn, immediately placed in microfuge tubes and kept under similar light and temperature conditions. Small amounts of ¹⁴C—CO₂ or ¹⁴C—HCO₃⁻ which did not affect the final Ci concentration, were injected, and the Ci uptake terminated after 5 seconds by centrifugation.

General DNA Manipulations:

Genomic DNA was isolated as described elsewhere [12]. Standard recombinant DNA techniques were used for cloning and Southern analyses [12-13] using the Random Primed DNA Labeling Kit or the DIG system (Boehringer, Mannheim). Sequence analysis was performed using the Dye Terminator cycle sequencing kit, ABI Prism (377 DNA sequencing Perkin Elmer). The genomic library used herein was constructed using a Lambda EMBL3/BamHI vector kit available from Stratagene (La Jolla, Calif.).

Construction and Isolation of Mutant IL-2:

A modification of the method developed by Dolganov and Grossman [14] was used to raise and isolate new high-CO₂-requiring mutants [4, 5]. Briefly, genomic DNA was digested with TaqI and ligated into the AccI site of the polylinker of a modified Bluescript SK plasmid. The bluescript borne gene for conferring ampicillin resistance was inactivated by the insertion of a cartridge encoding kanamycin resistance (Kan^{r}, [8]) (within the ScaI site). Synechococcus sp. strain PCC 7942 cells were transfected with the library [12]. Single crossover events conferring Kan^{r} led to inactivation of various genes. The Kan^{r} cells were exposed to low CO₂ conditions for 8 hours for adaptation, followed by an ampicillin treatment (400 μg/mL) for 12 hours. Cells capable of adapting to low CO₂ and thus able to grow under these conditions were eliminated by this treatment. The high-CO₂-requiring mutant, IL-2, unable to divide under low CO₂ conditions, survived, and was rescued following the removal of ampicillin and growth in the presence of high CO₂ concentration.

Cloning of the Relevant Impaired Genomic Region from Mutant IL-2:

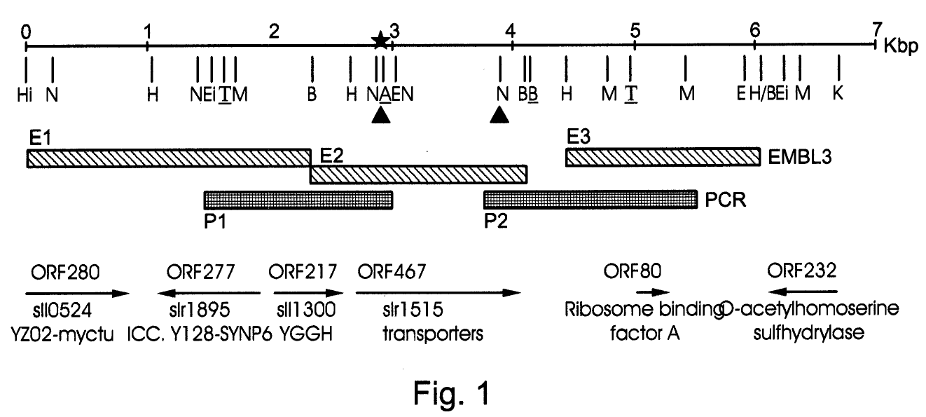

DNA isolated from the mutant was digested with ApaI located on one side of the AccI site in the polylinker; with BamHI or EcoRI, located on the other side of the AccI site; or with MfeI that does not cleave the vector or the Kan^{r} cartridge. These enzymes also cleaved the genomic DNA. The digested DNA was self-ligated followed by transfection of competent E. coli cells (strain DH5α). Kan^{r} colonies carrying the vector sequences bearing the origin of replication, the Kan^{r} cartridge and part of the inactivated gene were then isolated. This procedure was used to clone the flanking regions on both sides of the vector inserted into the mutant. A 1.3 Kbp ApaI and a 0.8 Kbp BamHI fragments isolated from the plasmids (one ApaI site and BamHI site originated from the vector's polylinker) were used as probes to identify the relevant clones in an EMBL3 genomic library of a wild type genome, and for Southern analyses. The location of these fragments in the wild type genome (SEQ ID NO: 1) is schematically shown in FIG. 1. The ApaI fragment is between positions 1600 to 2899 (of SEQ ID NO:1), marked as T and A in FIG. 1; the BamHI fragment is between positions 4125 to 4957 (of SEQ ID NO:1) marked as B and T in FIG. 1. The 0.8 Kbp BamHI fragment hybridized with the 1.6 Kbp HincII fragment (marked E3 in FIG. 1). The 1.3 Kbp ApaI fragment hybridized with an EcoRI fragment of about 6 Kbp. Interestingly, this fragment could not be cloned from the genomic library into E. coli. Therefore, the BamHI site was used (position 2348, SEQ ID NO: 1, FIG. 1) to split the EMBL3 clone into two clonable fragments of 4.0 and 1.8 Kbp (E1 and E2, respectively, E1 starts from a Sau3AI site upstream of the HindIII site positioned at the beginning of FIG. 1). Confirmation that these three fragments were indeed located as shown in FIG. 1 was obtained by PCR using wild type DNA as template, leading to the synthesis of fragments P1 and P2 (FIG. 1). Sequence analyses enabled comparison of the relevant region in IL-2 with the corresponding sequence in the wild-type.

Physiological Analysis of the IL-2 Mutant:

The IL-2 mutant grew nearly the same as the wild type cells in the presence of high CO₂ concentration but was unable to grow under low CO₂. Analysis of the photosynthetic rate as a function of external Ci concentration revealed that the apparent photosynthetic affinity of the IL-2 mutant was 20 mM Ci, which is about 100 times higher than the concentration of Ci at the low CO₂ conditions. The curves relating to the photosynthetic rate as a function of Ci concentration, in IL-2, were similar to those obtained with other high-CO₂-requiring mutants of Synechococcus PCC 7942 [16, 17]. These data suggested that the inability of IL-2 to grow under low CO₂ is due to the poor photosynthetic performance of this mutant.

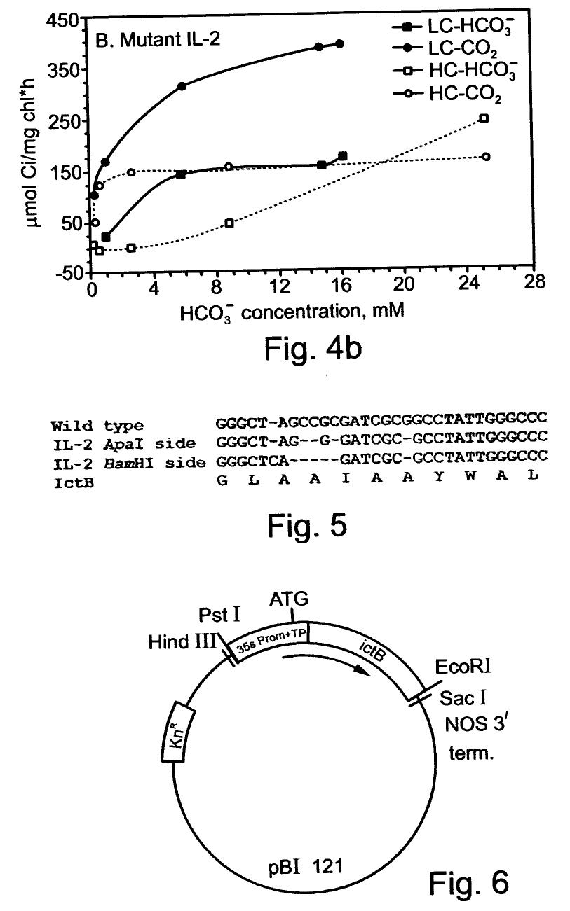

High-CO₂-requiring mutants showing such characteristics were recognized among mutants bearing aberrant carboxysomes [9, 10, 12, 18, 19] or defective in energization of Ci uptake [20, 21]. All the carboxysome-defective mutants characterized to date were able to accumulate Ci within the cells similarly to wild type cells. However, they were unable to utilize it efficiently in photosynthesis due to low activation state of rubisco in mutant cells exposed to low CO₂ [10]. This was not the case for mutant IL-2 which possessed normal carboxysomes but exhibited impaired HCO₃⁻ uptake (Table 1, FIGS. 4a-b). Measurements of ¹⁴Ci accumulation indicated that HCO₃⁻ and CO₂ uptake were similar in the high-CO₂-grown wild type and the mutant (Table 1).

TABLE 1

| | |
|---|---|
| CO₂ Uptake | HCO₃⁻ Uptake |

| | High CO₂ | Low CO₂ | High CO₂ | Low CO₂ |
|---|---|---|---|---|
| WT | 31.6 | 53.9 | 30.9 | 182.0 |
| IL-2 | 26.6 | 39.2 | 32.2 | 61.1 |

The rate of CO₂ and of HCO₃⁻ uptake in Synechococcus sp. PCC 7942 and mutant IL-2 as affected by the concentration of CO₂ in the growth medium. The unidirectional CO₂ or HCO₃⁻ uptake of cells grown under high CO₂ conditions or exposed to low CO₂ for 12 hours is presented in μmole Ci accumulated within the cells mg⁻¹ Chl h⁻¹. The results presented are the average of three different experiments, with four replicas in each experiment, the range of the data was within ±10% of the average. WT—wild type.

Uptake of HCO₃⁻ by wild type cells increased by approximately 6-fold following exposure to low CO₂ conditions for 12 hours. On the other hand, the same treatment resulted in only up to a 2-fold increase in HCO₃⁻ uptake for the IL-2 mutant. Uptake of CO₂ increased by approximately 50% for both the wild type and the IL-2 mutant following transfer from high- to low CO₂ conditions. These data indicate that HCO₃⁻ transport and not CO₂ uptake was impaired in mutant IL-2.

The Vₘₐₓ of HCO₃⁻ uptake, estimated by MIMS [7, 22] at steady state photosynthesis (FIG. 4a), were 220 and 290 μmol HCO₃⁻ mg⁻¹ Chl h⁻¹ for high- and low-CO₂-grown wild type, respectively, and the corresponding K_{1/2} (HCO₃⁻) were 0.3 and 0.04 mM HCO₃⁻, respectively. These estimates are in close agreement with those reported earlier [7]. In high-CO₂-grown mutant IL-2, on the other hand, the HCO₃⁻ transporting system was apparently inactive. The curve relating the rate of HCO₃⁻ transport as a function of its concentration did not resemble the expected saturable kinetics (observed for the wild type), but was closer to a linear dependence as expected in a diffusion mediated process (FIG. 4b). It was essential to raise the concentration of HCO₃⁻ in the medium to values as high as 25 mM in order to achieve rates of HCO₃⁻ uptake similar to the Vₘₐₓ depicted by the wild type.

The estimated Vmax of CO₂ uptake by high-CO₂-grown wild type and IL-2 was similar for both at around 130-150 μmol CO₂ mg⁻¹ Chl h⁻¹ and the K_{1/2}(CO₂) values were around 5 μM (FIGS. 4a-b), indicating that CO₂ uptake was far less affected by the mutation in IL-2. Mutant cells that were exposed to low CO₂ for 12 hours showed saturable kinetics for HCO₃⁻ uptake suggesting the involvement of a carrier. However, the K_{1/2} (HCO₃⁻) was 4.5 mM HCO₃⁻ (i.e., 15- and 100-fold lower than in high- and in low-CO₂-grown wild type, respectively) and the Vₘₐₓ was approximately 200 μmol HCO₃⁻ mg⁻Chl h⁻¹. These data indicate the presence of a low affinity HCO₃⁻ transporter that is activated or utilized following inactivation of a high affinity HCO₃⁻ uptake in the mutant. The activity of the low affinity transporter resulted in the saturable transport kinetics observed in the low-CO₂-exposed mutant. These data further demonstrated that the mutant was able to respond to the low CO₂ signal.

The reason for the discrepancy between the data obtained by the two methods used, with respect to HCO₃⁻ uptake in wild type and mutant cells grown under high-CO₂-conditions, is not fully understood. It might be related to the fact that in the MIMS method HCO₃⁻ uptake is assessed as the difference between net photosynthesis and CO₂ uptake [6, 7, 22]. Therefore, at Ci concentrations below 3 mM, where the mutant did not exhibit net photosynthesis, HCO₃⁻ uptake was calculated as zero (FIGS. 4a-b). On the other hand, the filtering centrifugation technique, as used herein, measured the unidirectional HCO₃⁻ transport close to steady state via isotope exchange, which can explain some of the variations in the results. Not withstanding, the data obtained by both methods clearly indicates severe inhibition of HCO₃⁻ uptake in mutant cells exposed to low CO₂. It is interesting to note that while the characteristics of HCO₃⁻ uptake changed during acclimation of the mutant to low CO₂, CO₂ transport was not affected (FIGS. 4a-b). It is thus concluded that the high-CO₂-requiring phenotype of IL-2 is generated by the mutation of a HCO₃⁻ transporter rather than in non-acclimation to low CO₂.

Genomic Analysis of the IL-2 Mutant:

Since IL-2 is impaired in HCO₃⁻ transport, it was used to identify and clone the relevant genomic region involved in the high affinity HCO₃⁻ uptake. FIG. 1 presents a schematic map of the genomic region in Synechococcus sp. PCC 7942 where the insertion of the inactivating vector by a single cross over recombination event (indicated by a star) generated the IL-2 mutant. Sequence analysis (GenBank, accession No. U62616, SEQ ID NO: 1) identified several open reading frames (identified in the legend of FIG. 1), some are similar to those identified in Synechocystis PCC 6803 [23]. Comparison of the DNA sequence in the wild type with those in the two repeated regions (due to the single cross over) in mutant IL-2, identified several alterations in the latter. This included a deletion of 4 nucleotides in the ApaI side and a deletion of 6 nucleotides but the addition of one bp in the BamHI side (FIG. 5). The reason(s) for these alterations is not known, but they occurred during the single cross recombination between the genomic DNA and the supercoiled plasmid bearing the insert in the inactivation library. The high-CO₂-requiring phenotype of mutant JR12 of Synechococcus sp. PCC 7942 also resulted from deletions of part of the vector and of a genomic region, during a single cross over event, leading to a deficiency in purine biosynthesis under low CO₂ [24].

The alterations depicted in FIG. 5 resulted in frame shifts which led to inactivation of both copies of ORF467 (nucleotides 2670-4073 of SEQ ID NO:1, SEQ ID NO:2) in IL-2. Insertion of a Kan^{r} cartridge within the EcoRV or NheI sites in ORF467, positions 2919 and 3897 (SEQ ID NO:1), respectively (indicated by the triangles in FIG. 1), resulted in mutants capable of growing in the presence of kanamycin under low CO₂ conditions, though significantly (about 50%) slower than the wild type. Southern analyses of these mutants clearly indicated that they were merodiploids, i.e., contained both the wild type and the mutated genomic regions.

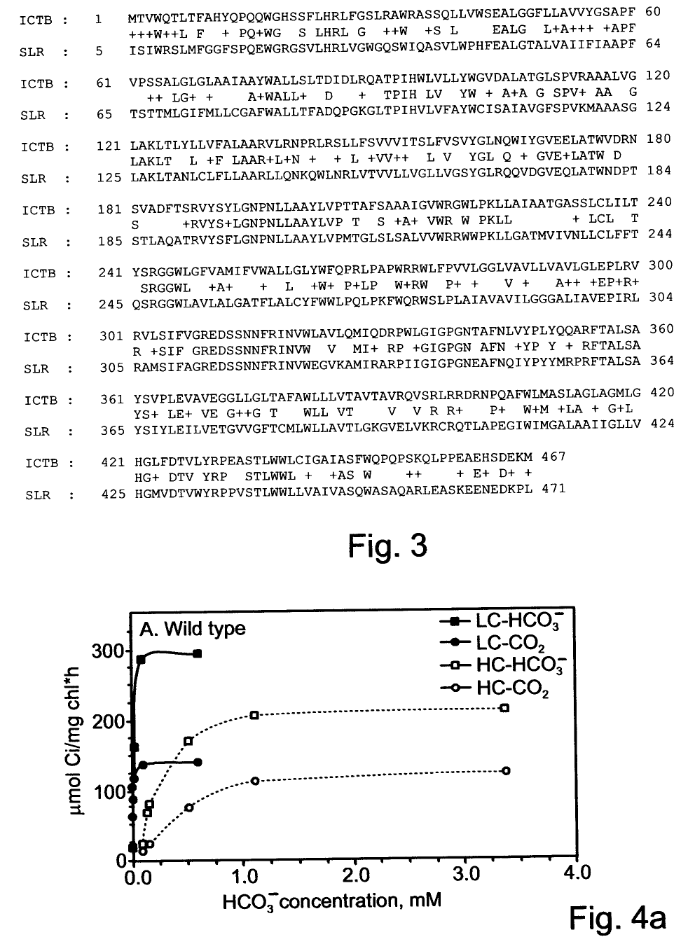

FIGS. 2 and 3 show nucleic and amino acid alignments of ictB and slr1515, the most similar sequence to ictB identified in the gene bank, respectively. Note that the identical nucleotides shared between these nucleic acid sequences (FIG. 2) equal 56%, the identical amino acids shared between these amino acid sequences (FIG. 3) equal 47%, the similar amino acids shared between these amino acid sequences (FIG. 3) equal 16%, bringing the total homology therebetween to 63% (FIG. 3). When analyzed without the transmembrane domains, the identical amino acids shared between these amino acid sequences equal 40%, the similar amino acids shared between these amino acid sequences equal 12%, bringing the total homology therebetween to 52%.

### Example 2

### ictB—a Putative Inorganic Carbon Transporter

The protein encoded by ORF467 (SEQ ID NO:3) contains 10 putative transmembrane regions and is a membrane integrated protein. It is somewhat homologous to several oxidation-reduction proteins including the Na⁺/pantothenate symporter of E. coli (Accession No. P16256). Na⁺ ions are essential for HCO₃⁻ uptake in cyanobacteria and the possible involvement of a Na⁺/HCO₃⁻ symport has been discussed [3, 25, 26]. The sequence of the fourth transmembrane domain contains a region which is similar to the DCCD binding motif in subunit C of ATP synthase with the exception of the two outermost positions, replaced by conservative changes in ORF467. The large number of transport proteins that are homologous to the gene product of ORF467 also suggest that it is also a transport protein, possibly involved in HCO₃⁻ uptake. ORF467 is referred to herein as ictB (for inorganic carbon transport B [27]).

Sequence similarity between cmpA, encoding a 42-kDa polypeptide which accumulates in the cytoplasmic-membrane of low-CO₂-exposed Synechococcus PCC 7942 [28], and nrtA involved in nitrate transport [29], raised the possibility that CmpA may be the periplasmic part of an ABC-type transporter engaged in HCO₃⁻ transport [21, 42]. The role of the 42 kDa polypeptide, however, is not clear since inactivation of cmpA did not affect the ability of Synechococcus PCC7942 [30] and Synechocystis PCC6803 [21] to grow under a normal air level of CO₂ but growth was decreased under 20 ppm CO₂ in air [21]. It is possible that Synechococcus sp. PCC 7942 contains three different HCO₃⁻ carriers: the one encoded by cmpA; IctB; and the one expressed in mutant IL-2 cells exposed to low CO₂ whose identity is yet to be elucidated. These transporters enable the cell to maintain inorganic carbon supply under various environmental conditions.

### Example 3

### Transgenic Plants Expressing ictB

The coding region of ictB was cloned downstream of a strong promoter (CaMV 35S) and downstream to, and in frame with, the transit peptide of pea rubisco small subunit. This expression cassette was ligated to vector sequences generating the construct shown in FIG. 6.

Arabidopsis thaliana and tobacco plants were transformed with the expression cassette described above using the Agrobacterium method. Seedlings of wild type and transgenic Arabidopsis plants were germinated and raised for 10 days under humid conditions. The seedlings were then transferred to pots, each containing one wild type and three transgenic plants. The pots were placed in two growth chambers (Binder, Germany) and grown at 20-21° C., 200 micromol photons m⁻² sec⁻¹ (8 h:16 h, light:dark). The relative humidity was maintained at 25-30% in one growth chamber and 70-75% in the other. In growth experiments, the plants were harvested from both growth chambers after 18 days of growth. The plants were quickly weighed (fresh weight) and dried in the oven overnight in order to determine the dry weight.

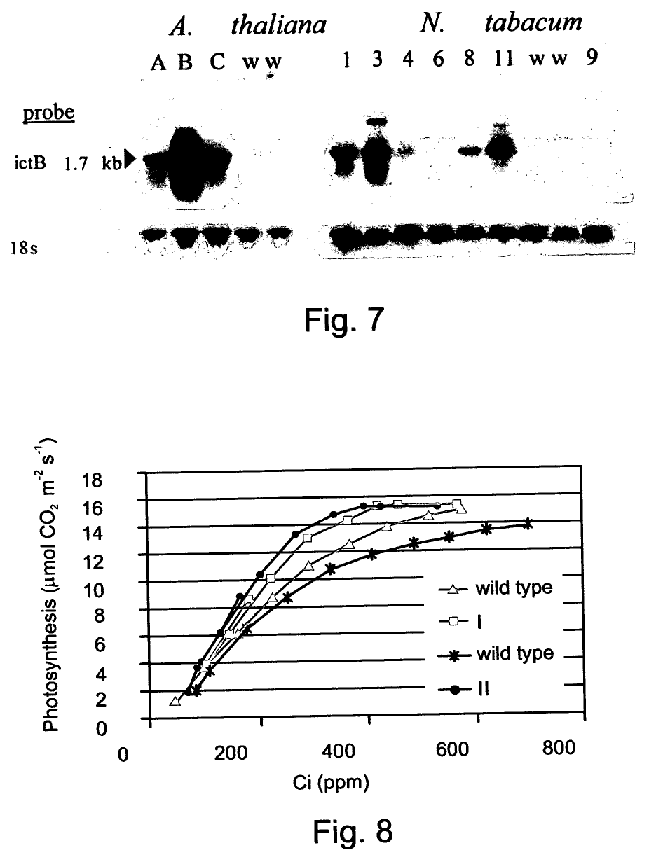

Northern analysis of plant RNA demonstrated that levels of ictB mRNA varied between different transgenic plants, while as expected, ictB mRNA was not detected in the Wild type plants (FIG. 7).

Measurements of the photosynthetic characteristics with respect to CO₂ concentration showed that in both tobacco (FIG. 8) and Arabidopsis (not shown) the rate of photosynthesis at saturating CO₂ level was similar in the transgenic and wild type plants. On the other hand, under air levels of CO₂ or lower (such as experienced under water stress when the stomata are closed) the transgenic plants exhibited significantly higher photosynthetic rates than the wild type (FIG. 8). Note that the slope of the curve relating photosynthesis to intercellular CO₂ concentration was steeper in the transgenic plants suggesting that the activity of Rubisco was higher in the transgenic plants.

### Example 4

### Growth Rate and Shift in Compensation Point of ictB Transgenic Plants

Materials and Methods

Measurements of photosynthetic rate and CO₂ compensation point: CO₂ and water vapor exchange were determined with the aid of a Li-Cor 6400 operated according to the instructions of the manufacturer (Li-Cor, Lincoln, Nebr.). Saturating light intensities of 750 and 500 μmol photons m⁻² s⁻¹ were used during the measurements with tobacco and Arabidopsis, respectively. The CO₂ compensation point was deduced from measurements of the rate of CO₂ exchange as affected by a range (0-150 μmole CO₂ L⁻¹) of CO₂ concentrations. The point of zero net exchange, i.e. the CO₂ concentration where the curve relating net CO₂ exchange to concentration crossed zero CO₂, represents the compensation point.

Results

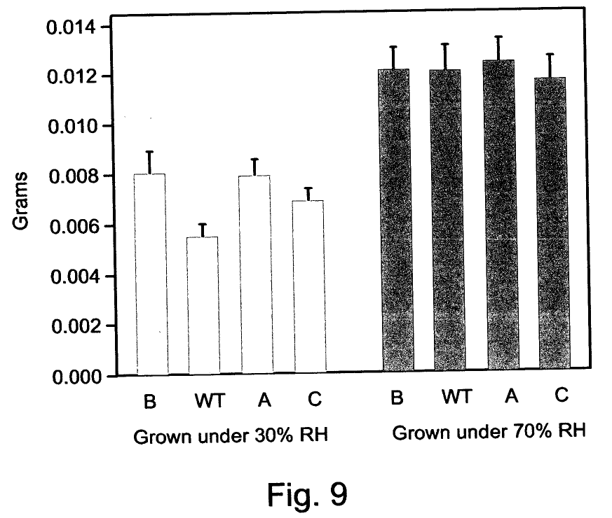

In view of the positive effect of ictB expression on photosynthetic performance, the transgenic plants of the present invention were further tested for growth rates as compared to wild type plants (FIG. 9).

Growth was faster in plants well supplied with water, maintained under the high (above 70%) relative humidity. Under such optimal conditions there was no significant difference between the wild type and the transgenic plants.

Surprisingly, however, the transgenic Arabidopsis plants grew significantly faster than the wild type under conditions of restricted water supply and low (lower than 40%) humidity (FIG. 9). These data demonstrated the ability of ictB to raise plant productivity particularly under growth limiting (dry) conditions where stomatal closure may lead to lower intercellular CO₂ level and thus growth retardation.

The significant effect of ictB expression on growth in growth limiting conditions can be due to elevated CO₂ concentration at the site of Rubisco in the transgenic plants, resulting from enhanced HCO₃⁻ entry to the chloroplasts. Such enhanced HCO₃⁻ transport would be expected to lower the compensation point for CO₂ and to lower the delta ¹³C of the organic matter produced [31]. Table 2 shows that the compensation point (point of zero net CO₂ exchange, a sensitive measure of photosynthetic capacity) measured in the transgenic plants was consistently lower than in the wild type controls (greater than 10% lower in Arabidopsis, and greater than 15% lower in the transgenic tobacco). The slope of the curve relating photosynthesis to intercellular CO₂ concentration (FIG. 8) was steeper in the transgenic plants suggesting (according to accepted models of photosynthesis [31-33]) that the activity of RubisCO in the plants expressing ictB was higher than in the wild type.

TABLE 2
The CO₂ compensation point in wild type and
transgenic Arabidopsis and tobacco plants

| | CO₂ Compensation |
|---|---|
| PLANT | point (μl/l) |
| Arabidopsis | |
| A | 39.2 ± 1.0 |
| B | 41 ± 1.1 |
| WILD TYPE | 46.1 ± 1.1 |
| Tobacco | |
| 3 | 47.1 ± 1.4 |
| 11 | 48 ± 1.6 |
| WILD TYPE | 56.9 ± 1.6 |

Taken together, these results indicate enhanced CO₂ concentrating capacity of the transgenic plants expressing ictB, most apparent under conditions of limited CO₂ supply, such activity most likely responsible for the increase in RubisCO activity in the transgenic plants.

### Example 5

### Enhanced Rubisco Activity in ictB Transgenic Plants

Materials and Methods

Measurements of RubisCO activity: The plants were grown for 18 days under low or high relative humidity with temperature and light conditions as above. They were placed at a similar distance and orientation from the light sources to minimize possible differences between them due to unequal local conditions. The leaves were excised 3 hours after the onset of illumination and immersed immediately in liquid nitrogen. Fifteen cm² of frozen leaves were ground in a buffer containing 1.5% PVP, 0.1% BSA, 1 mM DTT, protease inhibitors (Sigma) and 50 mM Hepes-NaOH pH 8.0. For in vitro activation, the extracts were centrifuged and aliquots of the supernatants were supplemented with 10 mM NaHCO₃ and 5 mM MgCl₂ (Badger and Lorimer, 1976) and maintained for at least 20 min. at 25° C. RubisCO activity was determined, either immediately or after the activation (Marcus and Gurevitz, 2000) in the presence of 20-150 μM ¹⁴CO₂ (6.2-9.3 Bq nmole⁻¹). The reaction was terminated after 1 min. by 6 N acetic acid and the acid stable products were counted in a scintillation counter (Marcus and Gurevitz, 2000). Time course analyses indicated that the RubisCO activities were constant for 1 min. and declined thereafter probably due to accumulation of inhibitory intermediate metabolites (Edmondson et al., 1990; Cleland et al., 1998; Kane et al., 1998). Quantification of the amount of RubisCO active sites was performed as in Marcus and Gurevitz (2000).

Results:

In addition to the sensitivity of the activity of RubisCO in photosynthetic plants to CO₂ concentration, the activation state of RubisCO in photosynthetic plants is highly sensitive to CO₂ concentration in close proximity to the enzyme. In order to determine whether expression of the ictB gene in transgenic plants results in increased RubisCO activity, transgenic and control plants were grown under an identical regimen of light, temperature and humidity for 18 days, and RubisCO activity measured in leaves in the activated (in vitro, maximal activity) and non-activated (in vivo, native activity) state.

TABLE 3
RubisCO activity in wild type (WT) and transgenic
tobacco plant grown under high humidity

| | RubisCO activity |
|---|---|
| Plant | (nmol C fixed/nmol catalytic site/m |
| WT, in vitro | 105 +/− 7 |
| Transgenic, in vitro | 103 +/− 8 |
| WT, in vivo | 84 +/− 7 |
| Transgenic, in vivo | 86 +/− 6 |

RubisCO activity was determined with (in vitro) or without (in vivo) prior activation. The reaction was terminated after 1 min. Other conditions as described in Materials and Methods procedures. n = 6.

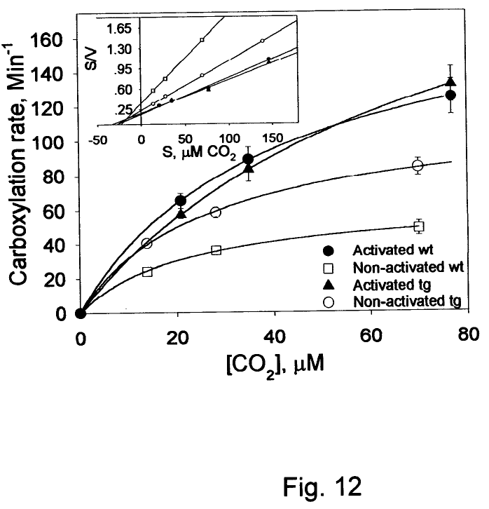

Surprisingly, under the growth limiting conditions (low humidity), the in vivo activity of RubisCO was about 40% higher in the transgenic than in the wild type plants over the entire range of CO₂ concentrations examined in the activity assays (FIG. 12). In contrast, following activation in vitro by the addition of CO₂ and MgCl₂, where RubisCO activity was close to its maximum, no significant difference was observed between the activities of wild type and transgenic plants maintained in either the humid (Table 3) or the dry conditions (FIG. 12), confirming that insertion of ictB did not alter the intrinsic properties of RubisCO. Under the humid conditions, the RubisCO activity observed without in vitro activation (most likely closely resembling those in vivo just before the leaves were immersed in liquid nitrogen) was about 85% that of the in vitro activated enzyme in both the wild type and the transgenic plants (Table 3).

The activities of RubisCO at increasing CO₂ concentrations is shown in FIG. 12 in order to emphasize the consistency of the data, even at various CO₂ levels, rather than to provide a complete account of the kinetic parameters of activated and non-activated RubisCO from tobacco. Nevertheless, analysis of the kinetic parameters from experiments similar to that depicted in FIG. 12, performed with the wild type and transgenic line 3 indicates that while the substrate affinity [Km(CO₂)] was scarcely affected by the expression of ictB, the Vmax of carboxylation, in vivo, was significantly enhanced by ictB expression in the transgenic plants. The higher in vivo RubisCO activity in the transgenic plants as compared with wild type controls (FIG. 12), under the growth limiting (dry) conditions where stomatal conductance may limit CO₂ supply, is consistent with the steeper slope of the curve relating photosynthetic rate to intercellular CO₂ concentration (FIG. 8). It will be noted that the in vivo RubisCO activities were lower than those depicted by the in vitro activated enzyme (FIG. 12, Table 3). This reduced in vivo RubisCO activity in the growth limiting (dry) vs. the high humidity-grown wild type control plants is possibly due to lower internal CO₂ concentration imposed by the decreased stomatal conductance. Significantly, it is under such growth-limiting conditions that the transgenic plants expressing the ictB gene exhibit enhanced photosynthesis and growth.

Thus, applying the teachings of the present invention one can transform plants such as C3 plants including, but not limited to, tomato, soybean, potato, cucumber, cotton, wheat, rice, barley and C4 crop plants, including, but not limited to, corn, sugar cane, sorghum and others, to thereby generate plants and crops having enhanced growth, and produce higher crop yield especially under limiting CO₂ and/or water limiting conditions.

### Example 5

### ictB Homologues

The phenomenon of acclimation to low CO₂ conditions is widespread in photosynthetic organisms, including many species of cyanobacteria. The CO₂ concentrating mechanisms enables these organisms to raise the CO₂ level at the carboxylating sites to overcome the large difference between the Km (CO₂) of RubisCO and the ambient dissolved CO₂ concentration. However, the mechanisms specifically responsible for enhanced CO₂ uptake in these species have yet to be elucidated. In order to determine whether ictB or ictB functional homologues are involved in similar CO₂ concentrating mechanisms in other species, proteins having amino acid sequence homology were identified from protein and nucleic acid sequence data banks.

Amino acid sequence homology, alignment and domain homology was derived using the InterProScan Program (www.ebi.ac.uk) and the CLUSTALW multiple alignment program. Genes highly homologous to ictB from Synechococcus PCC 7942 were found in all the cyanobacteria genomes for which a complete sequence analysis is available. One example of such homology is shown in FIGS. 10a and b, representing the hydropathy plots of ictB (FIG. 10a) and an homologous protein (Synwh0268) identified from the marine Synechococcus sp, Strain WH 8102 (FIG. 10b). Hydropathy analyses were performed using the TopPred program (http://bioweb.pasteur.fr/cgi-bin/seqanal/toppred.pl). The hydropathy plots identify 10 highly conserved regions of high hydrophobic value, indicating transmembrane domains, and a large region of high hydrophilicity, indicating a cytosolic and/or catalytic region.

FIG. 11 shows multiple alignments of amino acid sequences from 8 highly homologous genes identified from different cyanobacteria species. The sequences represent the proteins (from top to bottom) Anabaena, gene product of al15073 from Anabaena sp. strain PCC7120 (SEQ ID NO:6); Nostoc, Npun1329 from Nostoc punctiforme (SEQ ID NO:7); Trichodesmium, a putative gene product from Trichodesmium erythraeum IMS101(SEQ ID NO:10); SLR1515, gene product of slr1515 from Synechocystis sp. strain PCC 6803 (SEQ ID NO:5); IctB, gene product of ictB from Synechococcus sp. strain PCC 7942 (SEQ ID NO: 3), Thermosyn, tlr2249 from Thermosynechococcus elongatus (SEQ ID NO: 11); Prochloroco., Pmit1577 from Prochlorococcus marinus strain MIT 9313 (SEQ ID NO:12); and Synechococcus, Synwh0268 from the marine Synechococcus sp. strain WH 8102 (SEQ ID NO:13). Comparison of the overall homology indicates a very high level of sequence conservation (>70%), as demonstrated for the three ictB homologues from Synechocystis sp. PCC 6803, Anabaena PCC7120 and Nostoc punctiforme, shown in Table 5.

Comparison of membrane topology shows that all the proteins have similar hydrophobic (transmembrane) regions exhibiting high levels of identity and similarity [red star represents identity, green (colon) strong similarity and blue (dot) similarity]. Architecture analysis of the 8 proteins performed with the SMART TMHMM2 program (http://smart.heidelberg-emblde) also indicates high degree of homology within the conserved hydrophobic, transmembrane domains. Table 4 shows one example of such a comparison, between homologous ictB and Anabaena proteins.

TABLE 4
Confidently predicted domains, repeats, motifs and features:

| | | |
|---|---|---|
| DOMAIN TYPE | begin | end |

ictB

| | | |
|---|---|---|
| transmembrane | 39 | 61 |
| transmembrane | 65 | 82 |
| transmembrane | 95 | 112 |
| transmembrane | 116 | 138 |
| transmembrane | 145 | 167 |
| transmembrane | 198 | 217 |
| transmembrane | 224 | 241 |
| transmembrane | 245 | 264 |
| transmembrane | 276 | 298 |
| transmembrane | 363 | 385 |
| transmembrane | 406 | 428 |

Anabaena (all5073 from Anabaena)

| | | |
|---|---|---|
| transmembrane | 48 | 82 |
| transmembrane | 95 | 117 |
| transmembrane | 122 | 144 |
| transmembrane | 151 | 169 |
| transmembrane | 204 | 223 |
| transmembrane | 230 | 247 |
| transmembrane | 251 | 273 |
| transmembrane | 280 | 302 |
| low complexity | 338 | 345 |
| transmembrane | 369 | 391 |
| transmembrane | 411 | 430 |
| transmembrane | 440 | 457 |

Of great significance is the highly conserved hydrophilic region delineated by amino acid coordinates 308-375 of ictB (SEQ ID NO: 3) (FIG. 11), having surprisingly high homology between the various gene products (46.3% identity, 20.9% similarity, 67.2% total homology). Such high homology in a hydrophilic (catalytic) region spanning 72 amino acids is clearly a very strong indication that these proteins constitute a family of homologues having a similar function, that can also be used to transform plants in order to achieve the growth or yield enhancement described hereinabove. Two additional amino acid sequences from cyanobacteria exhibiting functional similarity and 75-80% homologous to ictB are listed in Table 5 below.

TABLE 5
Sequence homology between ictB and
amino acid sequences from Synechocystis
sp. PCC 6803, Anabaena PCC7120 and Nostoc punctiforme

| | Protein sequence | Polynucleotide sequence |
|---|---|---|
| Organism | SEQ ID NO: | SEQ ID NO: |
| Anabaena | 6 | 8 |
| PCC7120 | | |
| Nostoc | 7 | 9 |
| punctiforme | | |

| | | | | Weakly | Overall |
|---|---|---|---|---|---|
| | Putative/ | Identical | Similar | similar | homology |
| | charac. | amino | amino | amino | amino |
| Organism | function | acids % | acids % | acids % | acids % |
| Synechocystis | none | 46.41 | 19.41 | 10.13 | 75.95 |
| slr1515 | | | | | |
| Anabaena | none | 51.37 | 18.32 | 9.68 | 79.37 |
| PCC7120 | | | | | |
| Nostoc | none | 50.84 | 18.28 | 11.55 | 80.67 |
| punctiforme | | | | | |

### Expected Commercial Significance

On the basis of the enhanced photosynthesis, RubisCO activity and reduction in CO₂ compensation point resulting from expression of ictB in transgenic Arabidopsis and tobacco plants (see Examples 3 and 4 hereinabove), it is expected that expression of ictB in important commercial crop plants such as: wheat, rice, barley, potato, cotton, soybean, lettuce and tomato will lead to a significant and previously unattainable increase in growth and commercial yield of the transgenic crops. Most importantly, the enhanced growth of transgenic plants and crops of the present invention demonstrated under growth limiting conditions can provide substantially improved crop yields in regions where commercial cultivation of food crops is substantially inhibited by sub-optimal growth conditions, such as, for example, the arid growth conditions characterizing regions in Africa.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

### References

1. Kaplan, A., Schwarz, R., Lieman-Hurwitz, J. and Reinhold, L. (1991) Plant Physiol. 97, 851-855.

2. Badger, M. R. and Price, D. G. (1994) Ann. Rev. Plant Physiol. Plant Mol. Biol. 45, 369-399.

3. Kaplan, A., Schwarz, R., Lieman-Hurwitz, J., Ronen-Tarazi, M. and Reinhold, L. (1994) in: The Molecular Biology of the Cyanobacteria (Bryant, D. Ed.), pp. 469-485, Kluwer Academic Pub., Dordrecht, The Netherlands.

4. Ronen-Tarazi, M., Schwarz, R., Bouevitch, A., Lieman-Hurwitz, J., Erez, J. and Kaplan, A. (1995) in: Molecular Ecology of Aquatic Microbes (Joint, I., Ed.), pp. 323-334. Springer-Verlag, Berlin.

5. Kaplan, A., Ronen-Tarazi, M., Zer, H., Schwarz, R., Tchemov, D., Bonfil, D. J., Schatz, D., Vardi, A., Hassidim, M. and Reinhold, L. (1998) Can. J. Bot. 76, 917-924.

6. Sultemeyer, D., Price, G. D., Bryant, D. A. and Badger, M. R. (1997) Planta 201, 36-42.

7. Sultemeyer, D., Klughammer, B., Badger, M. R. and Price, G. D. (1998) Plant Physiol. 116, 183-192.

8. Ronen-Tarazi, M., Shinder, V. and Kaplan, A. (1998) FEMS Microbiol Lett. 159, 317-324.

9. Sambrook, J., Fritsch, E. F. and Maniatis, T. (1989) Molecular Cloning. A Laboratory Manual. Cold Spring Harbor Laboratory Press, Cold Spring Harbor, N.Y.

10. Kaplan, A., Marcus, Y. and Reinhold, L. (1988) in: Methods in Enzymology (Packer, L. and Glazer, A. N., Eds.), pp. 534-539. Academic Press, New York.

11. Tchemov, D., Hassidim, M., Luz, B., Sukenik, A., Reinhold, L. and Kaplan, A. (1997) Curr. Biol. 7, 723-728.

12. Badger, M. R. and Price, G. D. (1992) Physiol. Plant. 84, 606-615.

13. Ronen-Tarazi, M., Lieman-Hurwitz, J., Gabay, C., Orus, M. and Kaplan, A. (1995) Plant Physiol. 108, 1461-1469.

14. Dolganov, N. and Grossman, A. (1993) J. Bacteriol. 175, 7644-7651.

15. Ronen-Tarazi, M., Bonfil, D. J., Schatz, D. and Kaplan, A. (1998) Can. J. Bot. 76, 942-948.

16. Marcus, Y., Schwarz, R., Friedberg, D. and Kaplan, A. (1986) Plant Physiol. 82, 610-612.

17. Schwarz, R., Friedberg, D., Reinhold, L. and Kaplan, A. (1988) Plant Physiol. 88, 284-288.

18. Fukuzawa, H., Suzuki, E., Komukai, Y. and Miyachi, S. (1992) Proc. Natl. Acad. Sci. USA 89, 4437-4441.

19. Schwarz, R., Reinhold, L. and Kaplan, A. (1995) Plant Physiol. 108, 183-190.

20. Marco, M., Ohad, N., Schwarz, R., Lieman-Hurwitz, J., Gabay, C. and Kaplan, A. (1993) Plant Physiol. 101, 1047-1053.

21. Ohkawa, H., Sonoda, M., Katoh, H. and Ogawa, T. (1998) in: Proceedings of The Third International Symposium on Inorganic Carbon Uptake by Aquatic Photosynthetic Organisms (Colman, B. Ed.), Can J Bot 76, 1035-1042.

22. Yu, J. W., Price, G. D. and Badger, M. R. (1994) Aust. J. Plant Physiol. 21, 185-195.

23. Kaneko, T., Sato, S., Kotani, H., Tanaka, A., Asamizu, E., Nakamura, Y., Miyajima, N., Hirosawa, M., Sugiura, M., Sasamoto, S., Kimura, T., Hosouchi, T., Matsuno, A., Muraki, A., Nakazaki, N., Naruo, K., Okumura, S., Shimpo, S., Takeuchi, C., Wada, T., Watanabe, A., Yamada, M., Yasuda, M. and Tabata, S. (1996) DNA Res. 3, 109-136.

24. Lieman-Hurwitz J., Schwarz, R., Martinez, F., Maor, Z., Reinhold, L., and Kaplan, A. (1990) in: Proceedings of The Second International Symposium on Inorganic Carbon Uptake by Aquatic Photosynthetic Organisms (Colman, B. Ed.), Can J Bot 69: 945-950.

25. Kaplan, A., Schwarz, R., Ariel, R., and Reinhold, L. (1990) in: Regulation of Photosynthetic Processes (Kanay, R., Katoh, R. S. and Miyachi, S. Eds.), Special Issue of the Botanical Magazine, vol. 2, pp. 53-71, Tokyo.

26. Tyrrell, P. N., Kandasamy, R. A., Crotty, C. M. and Espie, G. S. (1996) Plant Physiol. 112, 79-88

27. Ogawa, T. (1991) Plant Physiol. 96, 280-284.

28. Omata, T. and Ogawa, T. (1986) Plant Physiol. 80, 525-530.

29. Omata, T. (1992) in: Research in Photosynthesis (Murata, N. Ed.) vol. III, 807-810, Kluwer Academic Pub., Dodrecht, The Netherlands.

30. Omata, T., Carlson, T. J., Ogawa, T. and Pierce, J. (1990) Plant Physiol. 93, 305-311.

31 Woodrow, I. E. and Berry, J. A. (1988) Ann. Rev. Plant Physiol 39, 533-594.

32 Mott, K. A. and Woodrow, I. E. (2000) Journal of Experimental Botany 51, 399-406.

33 Poolman, M. G., Fell, D. A. and Thomas, S. (2000) Journal of Experimental Botany 51, 319-328.

34 Kaplan, A. and Reinhold, L. (1999) Annu. Rev. Plant Physiol. Plant Mol. Biol. 50, 539-570.

35 Spreitzer, R. J. (1999) Photosynthesis Research 60, 29-42.

36 Horton, P. (2000) Journal of Experimental Botany 51, 475-485.

37 Matsuoka, M., Nomura, M., Agarie, S., Miyao-Tokutomi, M. and Ku, M. S. B. (1998) Journal of Plant Research 111, 333-337.

38 Nomura, M. et al. (2000) Plant Mol. Biol. 44, 99-106.

39 Takeuchi, K., Akagi, H., Kamasawa, N., Osumi, M. and Honda, H. (2000) Planta 211, 265-274.

40 Suzuki, S., Murai, N., Burnell, J. N. and Arai, M. (2000) Plant Physiol. 124, 163-172.

41 Bonfil, D. J., Ronen-Tarazi, M., Sultemeyer, D., Lieman-Hurwitz, J., Schatz, D. and Kaplan, A. (1998) FEBS Lett. 430, 236-240.

42. Maeda, S- I., Price, G. D., Badger, M. R., Enomoto, C. and Omata, T. (2000) J. Biol. Chem. 275, 20551-20555

What is claimed is:

1. A method of obtaining plants characterized by enhanced growth and/or commercial yield under growth limiting conditions, the method comprising:
(a) obtaining a population of plants transformed to express a polypeptide having an amino acid sequence at least 60% homologous to that set forth in SEQ ID NOs: 3, 5, 6, 7, 10, 11, 12 or 13;
(b) growing said population of plants under the growth limiting condition to thereby detect plants of said population having enhanced growth and/or commercial yield; and
(c) selecting plants expressing said polypeptide having enhanced growth and/or commercial yield as compared to control plants,
thereby obtaining plants characterized by enhanced growth and/or commercial yield under growth limiting conditions.

2. The method of claim 1, wherein said amino acid sequence is as set forth by SEQ ID NOs: 3, 5, 6, 7, 10, 11, 12 or 13.

3. The method of claim 1, wherein step (a) is effected by transforming at least a portion of the plants of said population with a nucleic acid construct comprising a polynucleotide region encoding said polypeptide.

4. The method of claim 3, wherein said transforming is effected by a method selected from the group consisting of Agrobacterium mediated transformation, viral infection, electroporation and particle bombardment.

5. The method of claim 3, wherein said nucleic acid construct further comprises a second polynucleotide region encoding a transit peptide, said second polynucleotide being operably linked to said polynucleotide region encoding said polypeptide having an amino acid sequence at least 60% homologous to that set forth in SEQ ID NOs: 3, 5, 6, 7, 10, 11, 12 or 13.

6. The method of claim 3, wherein said nucleic acid construct further comprises a promoter sequence operably linked to said polynucleotide region encoding said polypeptide having an amino acid sequence at least 60% homologous to that set forth in SEQ ID NOs: 3, 5, 6, 7, 10, 11, 12 or 13.

7. The method of claim 6, wherein said nucleic acid construct further comprises a promoter sequence operably linked to both said polynucleotide region encoding said polypeptide having an amino acid sequence at least 60% homologous to that set forth in SEQ ID NOs: 3, 5, 6, 7, 10, 11, 12 or 13 and to said second polynucleotide region.

8. The method of claim 6, wherein said promoter is functional in eukaryotic cells.

9. The method of claim 6, wherein said promoter is selected from the group consisting of a constitutive promoter, an inducible promoter, a developmentally regulated promoter and a tissue specific promoter.

10. The method of claim 1, wherein said plants are C3 plants.

11. The method of claim 10, wherein said C3 plants are selected from the group consisting of tomato, soybean, potato, cucumber, cotton, wheat, rice, barley, lettuce, solidago, banana and poplar.

12. The method of claim 1, wherein said plants are C4 plants.

13. The method of claim 12, wherein said C4 plants are selected from the group consisting of corn, sugar cane and sorghum.

14. The method of claim 1, wherein said enhanced growth is a growth rate at least 10% higher than that of a control plant grown under similar growth conditions.

15. The method of claim 1, wherein said growth limiting condition is selected from the group consisting of water stress, low humidity, salt stress, and low CO₂ conditions.

16. The method of claim 15, wherein said low humidity is humidity lower than 40%.

17. The method of claim 15, wherein said low CO₂ (limiting conditions) is an intercellular CO₂ concentration lower than 10 micromolar.

18. The method of claim 14, wherein said growth rate is determined by at least one growth parameter selected from the group consisting of increased fresh weight, increased dry weight, increased root growth, increased shoot growth and increased flower development over time.

19. A transformed crop comprising a population of transformed plants expressing a polypeptide having an amino acid sequence at least 60% homologous to that set forth in SEQ ID NOs: 3, 5, 6, 7, 10, 11, 12 or 13 wherein each individual plant of said population is characterized by enhanced growth under limiting conditions as compared to similar non transformed plants when grown under at least one growth limiting condition.

20. The transformed crop of claim 19, wherein said amino acid sequence is as set forth by SEQ ID NOs: 3, 5, 6, 7, 10, 11, 12 or 13.

21. The transformed crop of claim 19, wherein said transformed plants are C3 plants.

22. The transformed crop of claim 21, wherein said C3 plants are selected from the group consisting of tomato, soybean, potato, cucumber, cotton, wheat, rice, barley, lettuce, solidago, banana, poplar and citrus.

23. The transformed crop of claim 19, wherein said transformed plants are C4 plants.

24. The transformed crop of claim 23, wherein said C4 plants are selected from the group consisting of corn, sugar cane and sorghum.

25. The transformed crop of claim 19, wherein a growth rate of said population of transformed plants is at least 10% higher than that of a population of similar non transformed plants when both are grown under a similar growth limiting condition.

26. The transformed crop of claim 25, wherein said growth rate is determined by at least one growth parameter selected from the group consisting of fresh weight, dry weight, root growth, shoot growth and flower development.

27. The transformed crop of claim 19, wherein said transformed plant is further characterized by an increased commercial yield as compared to similar non transformed plant grown under similar conditions.

28. The transformed crop of claim 19, wherein said at least one growth limiting condition is selected from the group consisting of water stress, low humidity, salt stress, and/or low CO₂ conditions.

29. The transformed crop of claim 28, wherein said low humidity is humidity lower than 40%.

30. The transformed crop of claim 28, wherein said low CO₂ is an intercellular CO₂ concentration lower than 10 micromolar.

31. A nucleic acid expression construct comprising:
(a) a first polynucleotide region encoding a polypeptide including an amino acid sequence at least 60% homologous to that set forth by SEQ ID NOs: 3, 5, 6, 7, 10, 11, 12 or 13; and
(b) a second polynucleotide region comprising a promoter sequence operably linked to said first polynucleotide region, said promoter sequence being functional in eukaryotic cells.

32. The nucleic acid expression construct of claim 31, wherein said promoter is selected from the group consisting of a constitutive promoter, an inducible promoter, a developmentally regulated promoter and a tissue specific promoter.

33. The nucleic acid expression construct of claim 31, wherein said promoter is a plant promoter.

34. The nucleic acid expression construct of claim 31, further comprising a second polynucleotide region encoding a transit peptide, said second polynucleotide being operably linked to said polynucleotide region encoding said polypeptide having an amino acid sequence at least 60% homologous to that set forth in SEQ ID NOs: 3, 5, 6, 7, 10, 11, 12 or 13.


SEQUENCE LISTING (13 sequences)

SEQ ID NO: 1 - DNA, 4957 bp, Synechococcus sp.
aagcttggattgaagcgatcggggtcaatcccagcgatgatcctcagttcctcctgatggtcgatccctttagcgccaagattgaggatctgctgcaagggctggatttcgcctatcccgaggccgtgaaagtgggcggattggccagtggtttgggggcagagtcagcgatcgccagcttgttttttcaagaccgacaggtcgatggcgtgattgggctagccctcagtggcaatgtccagctgcaggcgatcgtggctcagggctgtcgtccagttggcccgctttggcatgtggcagcggcggagcgcaacattctgcggcaacttcagaccgaagacgaggaaccgatcgccgcgctgcaagccctacagtcagtcctgcgtgatctctcccctgaattacagcgatcgctctgtgtgggcctggcctgcaattctttccaaacggtattacaaccgggcgacttcctgatccgtaacctgctggggtttgatccccgcactggtgctgtagcaatcggcgatcgcattcgagttgggcagcggctgcagctgcacgtacgggatgcccagacagcggcggatgacctcgagcggcaactggggcaatggtgccggcagcatgcgacaaaaccagcagcttccctcttgttttcctgcttggggcgcggcaagcccttctatcagcaggccaacttcgagtcgcaactgattcagcattacctctcagagctgcccctagctggctttttctgtaatggcgaaatcggcccgatcgctggcagcacctacctgcatggctacacatcggtgctggctttgctgtcggccaaaactcactagcgccagcgagacctgattgtcgatctgctgagcgcgactgtagcgctggaaataggcccggacctgagcaggcgcatcggccaagctgaccgtagtatcaccgtcagccacccccgcccagaaattccgcaacatcggcaggagagcgatcgcctccgcctccgataaattcaacggctcatgggtcaacaggcggatcaagtactctgactgcgatcgccatccattcccgccgaaaacgtttgtaaatcagtcttgatccggtagcgatcgcacccgacgggactctagttctagttgccaaccttcagcggcaggttgtacggttccgagtcggtagggatggggatagctgaccaaggaaccggtcgtgacttcccagagagcaccttgctgactggtggcttggatgtggaggtggcctgtgaagatcaccgagacgctgcccgcttcgaggattgatcgcaattcctcggcattttctaagatgtagcgctgaccaagcggatgctgctgttgatcgggcagatgctccaacacattgtggtgaatcatcacccagcgttggctagcggtggaagtggcgagttcttgttgcagccagttgagttgcgcgcaatcgactcgcccccgatgcagttgatggcccgcttcatcaaaagcgatcgaattcagcgcaaacagatcgagatccggtgcgatcgtgcagcgatagtaggggcgatcgctcgtgaagccaaagtcttgatagagctcgacaaactcggccacaccggtgcgatcgcgatcgctcgctgcggcgggcatatcgtggttgcccggcaccacatagaccggatagggcaactggcgcaattgttgcagcagccactgatggttttcccgctccccgtgctgggttaaatcccccggcagcaacaggaagtccaaatccagcgctgccagttctgtcaggatttgctcaaaagccggaatgctgcactcaatcaaatggaagcgatggggatggtgccaaattgtctgcggcagtccaatgtggagatcgctcagcagcgcaaatcgaaacgctcggttcattgccatcccctcagctatcgagcccgattctaggcgaagctaggtcgagtccgttgtcttcagttgcaagcattcatggccagagttcgcgttcggcagcacgtcaatccgctctctcagaaattccaagtggtcacgacttggccggattggcaacaggtctatgcggactgcgatcgcccgctgcatttggatattggctgtgctcgcgggcgctttctgctggcaatggcgacacgacaacctgagtggaattatctggggctggaaattcgtgagccgctggtagatgaggcgaacgcgatcgcccgcgaacgtgaactgaccaatctctactaccacttcagcaacgccaatttggacttggaaccgctgctgcgatcgctgccgacagggattttgcagcgggtcagcattcagttcccggatccttggttcaagaaacgccatcaaaagcgacgcgtcgtccagccggaactggtgcaagccctcgcgactgcgttacctgctggtgcagaggtctttctgcaatccgatgtgctggaagtgcaggcagagatgtgcgaacactttgcggcggaaccccgctttcagcgcacctgcttggactggctgccggaaaatccgctgcccgtcccgaccgagcgcgaaattgccgttcaaaacaaacagttgccagtctaccgtgctctcttcattcggcagccagcggactaagctcttaaggcaagcgttgacgcgatcgcgatgactgtctggcaaactctgacttttgcccattaccaaccccaacagtggggccacagcagtttcttgcatcggctgtttggcagcctgcgagcttggcgggcctccagccagctgttggtttggtctgaggcactgggtggcttcttgcttgctgtcgtctacggttcggctccgtttgtgcccagttccgccctagggttggggctagccgcgatcgcggcctattgggccctgctctcgctgacagatatcgatctgcggcaagcaacccccattcactggctggtgctgctctactggggcgtcgatgccctagcaacgggactctcacccgtacgcgctgcagctttagttgggctagccaaactgacgctctacctgttggtttttgccctagcggctcgggttctccgcaatccccgtctgcgatcgctgctgttctcggtcgtcgtgatcacatcgctttttgtcagtgtctacggcctcaaccaatggatctacggcgttgaagagctggcgacttgggtggatcgcaactcggttgccgacttcacctcacgggtttacagctatctgggcaaccccaacctgctggctgcttatctggtgccgacgactgccttttctgcagcagcgatcggggtgtggcgcggctggctccccaagctgctggcgatcgctgcgacaggtgcgagcagcttatgtctgatcctcacctacagtcgcggtggctggctgggttttgtcgccatgatttttgtctgggcgttattagggctctactggtttcaaccccgtctacccgcaccctggcgacgctggctattcccagtcgtattgggtggactagtcgcggtgctcttggtggcggtgcttggacttgagccgttgcgcgtgcgcgtgttgagcatctttgtggggcgtgaagacagcagcaacaacttccggatcaatgtctggctggcggtgctgcagatgattcaagatcggccttggctgggcatcggccccggcaataccgcctttaacctggtttatcccctctatcaacaggcgcgctttacggcgttgagcgcctactccgtcccgctggaagtcgcggttgagggcggactactgggcttgacggccttcgcttggctgctgctggtcacggcggtgacggcggtgcggcaggtgagccgactgcggcgcgatcgcaatccccaagccttttggttgatggctagcttggccggtttggcaggaatgctgggtcacggtctgtttgataccgtgctctatcgaccggaagccagtacgctctggtggctctgtattggagcgatcgcgagtttctggcagccccaaccttccaagcaactccctccagaagccgagcattcagacgaaaaaatgtagcgggctccccaacaaattcctgtgcacccgactggatccaccacctaaactggatcccaaaggtatccggtggatctagggtcataacgaactccgaccgcgatcgcgtccgcgaactgaacctccatcgcaccgaagcggagttcgttagtcgttgaagagccaatgctagagggggctgccgaagcagttgggctggaagcaggctgcgagaagccacccgcatccaaggcaaagttcagccgaccttccgcaaagactacgatcgccacggcggctctgccagctaagtcagcgctgggttagttgtcatagcagtccgcagacaagttaggacaacttcatagagggactcgctcagagtcaacagccgctgtccgtgggggtgcgcaatcacccccacacccacgcactgggggactcgactcccccaggccccccgcaacaagatttcggataaggggcatcggctgaatcgcgatcgctgcgggtaaaactagccggtgttagccatgggtttgagactaatcggcacggggcaaaacgtcctgatttatttgctcaatgtgataggttacatcgtcaaaaacaaggcccaagaggtaggaaaaatcacgaccgcccaagtccgagggctttgctgttgggagcgacctagggcagactagacagagcattgctgtgagccaaagcgccttcaattgctggcggctgtgggtttttcggaggttgccaaatgaaagaccttttcgtcaatgtcctccgctatccccgctacttcatcaccttccagctgggtattttttagtcgatctaccagtgggtgcggccgatggttcgcaacccagtcgcggcttgggcgctgctaggctttggagtttcga

SEQ ID NO: 2 - DNA, 1404 bp, Synechococcus sp.
atgactgtctggcaaactctgacttttgcccattaccaaccccaacagtggggccacagcagtttcttgcatcggctgtttggcagcctgcgagcttggcgggcctccagccagctgttggtttggtctgaggcactgggtggcttcttgcttgctgtcgtctacggttcggctccgtttgtgcccagttccgccctagggttggggctagccgcgatcgcggcctattgggccctgctctcgctgacagatatcgatctgcggcaagcaacccccattcactggctggtgctgctctactggggcgtcgatgccctagcaacgggactctcacccgtacgcgctgcagctttagttgggctagccaaactgacgctctacctgttggtttttgccctagcggctcgggttctccgcaatccccgtctgcgatcgctgctgttctcggtcgtcgtgatcacatcgctttttgtcagtgtctacggcctcaaccaatggatctacggcgttgaagagctggcgacttgggtggatcgcaactcggttgccgacttcacctcacgggtttacagctatctgggcaaccccaacctgctggctgcttatctggtgccgacgactgccttttctgcagcagcgatcggggtgtggcgcggctggctccccaagctgctggcgatcgctgcgacaggtgcgagcagcttatgtctgatcctcacctacagtcgcggtggctggctgggttttgtcgccatgatttttgtctgggcgttattagggctctactggtttcaaccccgtctacccgcaccctggcgacgctggctattcccagtcgtattgggtggactagtcgcggtgctcttggtggcggtgcttggacttgagccgttgcgcgtgcgcgtgttgagcatctttgtggggcgtgaagacagcagcaacaacttccggatcaatgtctggctggcggtgctgcagatgattcaagatcggccttggctgggcatcggccccggcaataccgcctttaacctggtttatcccctctatcaacaggcgcgctttacggcgttgagcgcctactccgtcccgctggaagtcgcggttgagggcggactactgggcttgacggccttcgcttggctgctgctggtcacggcggtgacggcggtgcggcaggtgagccgactgcggcgcgatcgcaatccccaagccttttggttgatggctagcttggccggtttggcaggaatgctgggtcacggtctgtttgataccgtgctctatcgaccggaagccagtacgctctggtggctctgtattggagcgatcgcgagtttctggcagccccaaccttccaagcaactccctccagaagccgagcattcagacgaaaaaatgtag

SEQ ID NO: 3 - protein, 467 aa, Synechococcus sp.
MTVWQTLTFAHYQPQQWGHSSFLHRLFGSLRAWRASSQLLVWSEALGGFLLAVVYGSAPFVPSSALGLGLAAIAAYWALLSLTDIDLRQATPIHWLVLLYWGVDALATGLSPVRAAALVGLAKLTLYLLVFALAARVLRNPRLRSLLFSVVVITSLFVSVYGLNQWIYGVEELATWVDRNSVADFTSRVYSYLGNPNLLAAYLVPTTAFSAAAIGVWRGWLPKLLAIAATGASSLCLILTYSRGGWLGFVAMIFVWALLGLYWFQPRLPAPWRRWLFPVVLGGLVAVLLVAVLGLEPLRVRVLSIFVGREDSSNNFRINVWLAVLQMIQDRPWLGIGPGNTAFNLVYPLYQQARFTALSAYSVPLEVAVEGGLLGLTAFAWLLLVTAVTAVRQVSRLRRDRNPQAFWLMASLAGLAGMLGHGLFDTVLYRPEASTLWWLCIGAIASFWQPQPSKQLPPEAEHSDEKM

SEQ ID NO: 4 - DNA, 1425 bp, Synechocystis sp.
atggtgtctcccatctctatctggcgatcgctgatgtttggcggtttttccccccaggaatggggccggggcagtgtgctccatcgtttggtgggctggggacagagttggatacaggctagtgtgctctggccccacttcgaggcattgggtacggctctagtggcaataatttttattgcggctcccttcacctccaccaccatgttgggcatttttatgctgctctgtggagccttttgggctctgctgacctttgctgatcaaccagggaagggtttgactcccatccatgttttagtttttgcctactggtgcatttcggcgatcgccgtgggattttctccggtaaaaatggcggcggcgtcggggttagcgaaattaacagctaatttatgtctgtttctactggcggcgaggttattgcaaaacaaacaatggttgaaccggttagtaaccgttgttttactggtagggctattggtggggagttacggtctgcgacaacaggtggacggggtagaacagttagccacttggaatgaccccacctctaccttggcccaggccactagggtatatagctttttaggtaatcccaatctcttggcggcttacctggtgcccatgacgggtttgagcttgagtgccctggtggtatggcgacggtggtggcccaaactgctgggagcaaccatggtgattgttaacctactctgtctcttttttacccagagccggggcggttggctagcagtgctggccctgggagctaccttcctggccctttgttacttctggtggttaccccaattacccaaattttggcaacggtggtctttgcccctggcgatcgccgtggcggttatattaggtgggggagcgttgattgcggtggaaccgattcgactcagggccatgagcatttttgctgggcgggaagacagcagtaataatttccgcatcaatgtttgggaaggggtaaaagccatgatccgagcccgccctatcattggcattggcccaggtaacgaagcctttaaccaaatttatccttactatatgcggccccgcttcaccgccctgagtgcctattccatttacctagaaattttggtggaaacgggtgtagttggttttacctgtatgctctggctgttggccgttaccctaggcaaaggcgtagaactggttaaacgctgtcgccaaaccctcgccccggaaggcatctggattatgggggctttagcggcgatcatcggtttgttggtccacggcatggtagatacagtctggtaccgtcccccggtgagcactttgtggtggttgctagtggccattgttgctagtcagtgggccagcgcccaggcccgtttggaggccagtaaagaagaaaatgaggacaaacctcttcttgcttcataa

SEQ ID NO: 5 - protein, 474 aa, Synechocystis sp.
MVSPISIWRSLMFGGFSPQEWGRGSVLHRLVGWGQSWIQASVLWPHFEALGTALVAIIFIAAPFTSTTMLGIFMLLCGAFWALLTFADQPGKGLTPIHVLVFAYWCISAIAVGFSPVKMAAASGLAKLTANLCLFLLAARLLQNKQWLNRLVTVVLLVGLLVGSYGLRQQVDGVEQLATWNDPTSTLAQATRVYSFLGNPNLLAAYLVPMTGLSLSALVVWRRWWPKLLGATMVIVNLLCLFFTQSRGGWLAVLALGATFLALCYFWWLPQLPKFWQRWSLPLAIAVAVILGGGALIAVEPIRLRAMSIFAGREDSSNNFRINVWEGVKAMIRARPIIGIGPGNEAFNQIYPYYMRPRFTALSAYSIYLEILVETGVVGFTCMLWLLAVTLGKGVELVKRCRQTLAPEGIWIMGALAAIIGLLVHGMVDTVWYRPPVSTLWWLLVAIVASQWASAQARLEASKEENEDKPLLAS

SEQ ID NO: 6 - protein, 475 aa, Anabaena PCC7120
MNLVWQRFTLSSLPLKQFLATSYLHRFLVGLLSSWRQTSFLLQWGDMIAAALLSLIYVLAPFVSSTLVGVLLIACVGFWLLLTLSDEPSSNNNSLVTPIHLLVLLYWGIAAVATALSPVKKAALTDLLTLTLYLLLFALCARVLRSPRLRSWIITLYLSASLVVSIYGMRQWRFGAPPLATWVDPESTLSKTTRVYSYLGNPNLLAGYLVPAVIFSLMAVFVWQGWARKSLAVTMLFVNTACLIFTYSRGGWIGLVVAVLGATALLVDWWSVQMPPFWRTWSLPILLGGLIGVLLIAVLFVEPVRFRVLSIFADRQDSSNNFRRNVWDAVFEMIRDRPIIGIGPGHNSFNKVYPLYQRPRYSALSAYSIFLEVAVEMGFVGLACFLWLIIVTINTAFVQLRQLRQSANVQGFWLVGALATLLGMLAHGTVDTIWFRPEVNTLWWLMVALIASYWTPLSANQCQELNLFKEEPTSN

SEQ ID NO: 7 - protein, 472 aa, Nostoc punctiforme
MNLVWQLFTLSSLPLKEYLATSYVHRSLVGLLSSWRQTSVLIQWGDAIAAVLLSSIYALAPFASSTLVGLLLVACVGFWLLLTLSDEVTPANVSSVTPIHLLVLLYWGIAVIATALSPVKKAALNDLGTLTLYLLLFALCARVLRSPRLRSWILTLYLHVSLIVSVYGLRQWFFGATALATWVDPESPLSKTTRVYSYLGNPNLLAGYLLPAVIFSLVAIFAWQSWLKKALALTMLIVNTACLILTFSRGGWIGLVVAVLAVMALLVFWKSVEMPPFWRTWSLPIVLGGLIGILLLAVIFVEPVRLRVFSIFADRQDSSNNFRRNVWDAVFEMIRDRPIFGIGPGHNSFNKVYPLYQHPRYTALSAYSILFEVTVETGFVGLACFLWLIIVTFNTALLQVRRLRRLRSVEGFWLIGAIAILLGMLAHGTVDTVWYRPEVNTLWWLIVALIASYWTPLTQNQTNPSNPEPAVN

SEQ ID NO: 8 - DNA, 1425 bp, Anabaena PCC7120
atgaatttagtctggcaacgatttactttatcttctttacctctaaaacagtttctagctacaagttacttacatcggttcctagtgggactgttatcttcttggcggcaaactagtttcttacttcagtggggagacatgattgcagctgcgttactcagcttgatatatgttttggctccctttgtctctagtactctcgttggtgtgctgctgatagcttgtgtaggtttttggttattgttgactttatctgatgaaccttcatcaaacaataactcccttgttactcccatacacctgttggtgttgctctattggggaattgctgctgtagcaacggcattatcaccagtcaagaaggcagcattaactgatttgttaaccttgactttgtatttgctactatttgctctttgtgccagggtgctgagatcgccgcgtctgaggtcttggatcattaccctctacctatctgcatcactggttgtcagtatatatggaatgcgacaatggcgttttggtgcgcccccactggcgacttgggttgatccagagtccaccttgtctaaaaccacaagggtttacagttatttaggcaatcccaatttgttggctggttatttagtaccggcggtgatttttagcctcatggcagtttttgtctggcagggctgggcaagaaaatctttagctgtaacaatgctgtttgtaaacactgcttgcctaatttttacttatagtcgtggcggctggattggtcttgtggtagcagtcttaggggcgacggcattgctagttgattggtggagtgtgcaaatgccgcctttttggcgaacctggtcattacccatacttttgggcggtttgatcggggtattgttgattgcggtgttatttgtcgagccagtccggtttcgagttctcagtatttttgccgatcgccaagatagcagcaataattttcgccgcaacgtgtgggatgctgtttttgagatgatccgcgatcgcccaattattggtattggccctggtcataattcttttaataaagtctaccctctttaccaaagacctcgttatagtgctttaagtgcctattccatcttcctagaggtggctgtagaaatgggttttgttggactagcttgctttctctggttaattatcgtcactattaatacagcattcgttcagctacgccaactgcgccaatctgccaatgtgcaaggattttggttggtgggtgccttagccacattgctgggaatgctggctcacggtacggtagacactatatggtttcgtccggaagttaatactctttggtggttaatggttgctctcattgctagctattggacacctttatccgcaaaccaatgtcaagaactcaatttatttaaggaagaacccacaagcaac

SEQ ID NO: 9 - DNA, 1419 bp, Nostoc punctiforme
atgaatttagtctggcaactatttactttatcatctttaccgctcaaagaatatcttgctaccagttacgtacaccgttctctggtgggactgttaagctcttggcggcaaaccagcgtcttgattcagtggggagatgcgatagcagctgtattactcagctcaatatatgcccttgcaccttttgcttcgagtactttggtaggtttattgctggtcgcttgtgtgggattttggctattgttgactttatctgatgaagtcacaccagcaaatgtctcgtcagtcactcccattcatctactggtattgctctactggggaattgccgtaatcgcaacagcattatcaccagtgaaaaaagcggcacttaacgacttgggaactttgaccttgtatttgctactatttgccctttgtgccagggtattaaggtcgcctcgcctccggtcttggattctcaccctttatctgcacgtatcgttaattgtcagtgtctatggattgcggcaatggttttttggagccacagcactggcaacttgggttgatccggaatctcctctgtctaagactacaagagtctacagttatttaggaaatcccaacttattggctggatacctcttaccagcagtaatttttagcttggtggcaatttttgcatggcaaagttggctcaaaaaagccttagcattaacaatgttgattgtcaatactgcctgcctgatcctgacttttagtcgtggcggttggattggactagtggtggcagttttggcggtgatggcattgctagttttttggaagagtgtggaaatgcctcctttttggcgtacttggtcgctgcccattgtcttaggaggtttaattgggatattactgttagcagtgatatttgtagagccagttcgcctgcgggtgttcagcatttttgctgaccgtcaagatagtagtaataattttcgtcgaaatgtgtgggatgctgtctttgagatgattcgcgatcgcccaattttcggtattggccctggtcacaactcttttaataaagtttatccgctctaccaacaccctcggtacactgctttaagtgcttattcgattttgtttgaagtgactgtagaaactgggtttgttggtttagcttgctttctctggctaataatcgtcacatttaatacggcgcttttgcaagtacgacgattgcgacgattgagaagtgtagagggattttggttaattggagcgatcgctattttgttgggtatgctcgctcacggcactgtagatactgtctggtatcgtcctgaagtcaataccctctggtggctcatcgttgctttaattgccagctactggacacctttaactcaaaaccagacaaatccatctaacccagaaccagcagtaaactaa

SEQ ID NO: 10 - protein, 461 aa, Trichodesmium erythraeum
MNSVWKKLTLTNLSFSDSEWLNASYLYGLLNGSLYNWRRGSWLMQWGEPLGFVLLAIVFTLAPFVNTTLIGFLLLASAGFWVLLKVSDNTQEYLTPIHLLIFLYWSIATLAVVISPAKTAAFSGWVKLTLYLLLFASGSLVLRSPRLRSWLINIYLLVSLVVSFYGIRQWIDKVEPLATWNDPTSAQAGATRVYSYLGNPNLLGGYLLPAIALSFVAIFAWSSWARKSLAVTILLVSCACLRYTGSRGSWIGFLALMFAMLILMWYWWRSYMPSFWQIWSLPIAVGSFAGLLILAVVLLEPLRDRVLSVFAGRQDSSNNFRMNVWMSVFDMIRDRPILGIGPGNDVFNKIYPLYQRPRYSALSSYSVPLEIVVETGFIGLTAFLWLLLVTFNQGVLQLKRLRDADNPQGYWLIGAIAAMVGLIGHGLVDTVWYRPQVNTIWWLMVAIIASYSSQQGVRSRE

SEQ ID NO: 11 - protein, 463 aa, Thermosynechococcus elongatus BP-1
MDVLLRRLDVEGWRSHSGVGRLLGLLQGWQEKSWLGRWLPSLAVLLVGLVLVLAPLMPSGMIGMLLAAGSGFWLLWTLAGEREGRWSGVHLLVLLYWGIALLATVLSPVPRAAMVGLGKLTLYLLFFALAERVMRNERWRSRLLTVYLLTALMVSVEGVRQWIFGAEPLATWTDPESALANVTRVYSFLGNPNLLAGYLLPSVPLSAAAIAVWQGWLPKLLAVVMLGMNAASLILTFSRGGWLGLVAATIAGVVLLGIWFWPRLPLQWRRWGVPTMGGLAIALCMGTIVSVPPLRERAASIFVARGDSSNNFRINVWMAVQQMIWARPWLGIGPGNVAFNQIYPLYQVNVRFTALGAYSIFLEILVEVGFIGFGVFLWLLAVLGDRARRCFEELRATGSPQGFWLMGTIAAMIGMLTHGLVDTIWFRPEVATLWWLMVAIVASFTPFQSKTANGTFSNRDPEP

SEQ ID NO: 12 - protein, 439 aa, Prochlorococcus marinus
MPKTAAPQPLLLRWQGHIPSSEAMQMRLQWIAGLLLMMLLATLPMLTRTGLGLTILAAGALWIIWGCVTPAGRIGSISSCLLVFFAIACLATGFSPVPLAAAKGLIKLISYLGVYALMRQLLATSSDWWDRLVAALLTGELISSVIAIRQLYAPAEEMAHWADPNSVAAGTVRIYGPLGNPNLLAGYLMPILPLALVALLRWQGLGAKLYAMVALGLGITATLFSFSRGGWLGMLSALAVILVLLLLRSTSHWPLVWRRLLPLIVIVLGTAMLVIAATQIEPIRTRITSLIAGRSDSSNNFRINVWLSSLEMIQARPWLGIGPGNAAFNRIYPLFQQPKFNALSAYSVPLEILVETGLAGLMASLALVITGMRKGLAGLNSNHPLALPALASLAAIAGLAVHGITDTIFFRPEVQLVGWFCLATLAQTQPEQKQLQQTE

SEQ ID NO: 13 - protein, 431 aa, Synechococcus  WH 8102
MADATDQRSIPLLLRWQGCLTPTASVQQRLELLSGVVLMLLLGSLPFVSRSGLGLELAAAGLLWLLWSLITPAKRLGAISRWVLLYLAIAWVCTGFSPVPIAAAKGLLKLTSYLGVYALMRTLLERQIVWWDRLLAALLGGGLFSSVLALRQLYASTDELAGWADPNSVSAGTIRIYGPLGNPNLLAGYLLPLVPLACIAVLRWKRLSCRLLAAVTALLAGSATVFTYSRGGWLGLLAALALAGMLILLRTTAHWPPLWRRLLPLAALLIAGIALALAITQLDPIRTRVLSLVAGRGDSSNNFRINVWLAAIEMVQDRPWLGIGPGNAAFNSIYPLYQQPKFDALSAYSVPLEILVETGIPGLLACLGLLLSSIQRGLRIHGQQGLIAIGSLAAIAGLLTQGITDTIFFRPEVQLIGWFALASLGATWLRD